SEQUENCE LISTING (5 sequences)

SEQ ID NO: 1 - DNA, 24 bp, artificial sequence
  primer
caatatgctgaaacgcgcgagaaa

SEQ ID NO: 2 - DNA, 22 bp, artificial sequence
  primer
cgctccatacatcttgaatgag

SEQ ID NO: 3 - DNA, 20 bp, artificial sequence
  primer
aagacattgatggcttttga

SEQ ID NO: 4 - DNA, 21 bp, artificial sequence
  primer
aagacgtaaatagcccccgac

SEQ ID NO: 5 - DNA, 24 bp, artificial sequence
  primer
aggactcgcaaaaacgtgatgaat

(19) United States
(12) Patent Application Publication
(10) Pub. No.: US 2009/0220948 A1
(43) Pub. Date: Sep. 3, 2009

(54) Methods and Device for Transmitting, Enclosing and Analysing Fluid Samples

(75) Inventors: Dominador Fortaleza Oviso, Singapore (SG); Dor Ngi Ting, Singapore (SG)

Correspondence Address: THELEN REID BROWN RAYSMAN & STEINER LLP, PO BOX 640640, SAN JOSE, CA, 95164-0640, US

(73) Assignee: ATTOGENIX BIOSYSTEMS PTE LTD., Singapore (SG)

(21) Appl. No.: 11/886,607

(22) PCT Filed: Mar. 16, 2005
(86) PCT No.: PCT/SG2005/000082

§ 371 Date: Aug. 1, 2008

(51) Int. Cl. C12Q 1/68; G01N 33/00; G01N 21/78; G01N 21/76; G01N 33/53; G01N 33/68; G01N 33/92; C12Q 1/02

(52) U.S. Cl. 435/6; 422/68.1; 436/164; 436/172; 435/7.9; 436/501; 436/94; 436/86; 436/89; 436/71; 435/29; 435/7.92

(57) ABSTRACT

A microfluidic device for analysing a fluid sample comprises at least one sample transmission channel; at least one multi-functional channel; and at least one reactor module which provides a fluid connection between the sample transmission channel and the multi-functional channel. Each reactor module comprises at least one reaction chamber, having at least one inlet in fluid communication with the sample transmission channel(s); and at least one fluid isolation chamber, which is in fluid communication with the outlet(s) of the reaction chamber(s). The fluid isolation chamber serves to isolate the fluid sample from the multi-functional channel(s).

### FIELD OF THE INVENTION

The present invention relates to a microfluidic device for transmitting, enclosing and analysing a fluid sample and a method of using the same.

### BACKGROUND OF THE INVENTION

“Lab-on-chips” are microdevices that integrate fluid manipulation functions to perform chemical and biochemical analysis processes. They miniaturize complex macro-scale chemical and biochemical analysis processes. They miniaturize complex macro-scale chemical or biochemical mixing, separation, reaction, analysis, detection and measurement processes. Miniaturisation by means of such microdevices, which are made of glass or polymeric substrates, minimizes the volumes of samples and reagents required as well as the time required for analysis. Such microdevices therefore offer advantages in terms of cost, speed and sample consumption. The term “Lab-on-chips” furthermore refers to the ability to integrate multiple samples and several steps of an analytical procedure, as well as potentially several assays into a single system of micro scale. “Lab-on-chips” have been applied to various methods, particularly in the field of life sciences. One such method comprises the use of enzymatic reactions including for instance the determination of kinetic constants (e.g. Burke, B J, Regnier, F E, Anal Chem (2003), 75, 1786-1791), the determination of analyte quantities (Wang, J, et al., Anal Chem (2001), 73, 1296-1300) or the polymerase chain reaction (‘PCR’, see e.g. Medintz, I L, et al., Electrophoresis, (2001), 22, 3845-3856). Other methods include capillary electrophoresis (Shao, X, et al., J. Microcolumn September, (1999), 11, 323-329), isoelectric focusing (Hofmann, 0, et al., Anal Chem, (1999), 71, 678-686) or immunoassays (e.g. Sato, K, et al., Electrophoresis, (2002), 23, 734-739).

One significant advantage of such systems is the increase in potential for automation and portability, thereby reducing the amount of hands-on labour and enabling on-site analysis and testing. At present however, the majority of microfluidic chips are micro scale devices coupled to a macro scale operational infrastructure. As an example, fluid transportation processes are often enabled through pumps and valves built in-situ or external to the microdevices. Micropumps and microvalves built in-situ to the system often require an additional driving force. Examples of such driving mechanisms for micropumps include check valve, peristaltic, rotary, centrifugal, ultrasonic, electro-hydrodynamic, electro-kinetic, phase transfer (which therefore requires temperature or pressure changes), electrowetting, magnetic or hydrodynamic mechanisms. Examples of such driving mechanisms for microvalves include pneumatic, thermopneumatic, thermomechanic, piezoelectric, electrostatic, electromagnetic, electrochemical and capillary mechanisms.

With the exception of capillary action, all of the above driving mechanisms either require the supply of external energy in form of e.g. electricity, magnetic fields, air pressure or thermal energy, or rely on mechanical parts that actuate the processes. Hence, these mechanisms depend on a peripheral macro scale operation infrastructure. Such peripheral macro scale supports hamper portability and thus nullify one of the advantages of microfluidic systems. It is therefore be desirable to use microdevices with self-distribution properties, which are independent of external devices and external power. Such devices have an improved portability and field deployability.

As mentioned above, capillary action provides a means of avoiding or reducing the dependency on peripheral macro scale support infrastructures through reducing the dependency on external forces as for instance electrical currents, mechanical forces, pressure changes, or temperature differences. It is therefore no surprise that they have been explored extensively to control and/or direct the flow of fluid. Capillary forces result from surface affinities between matters and depend on material properties such as their surface chemistry, surface morphology and structure. The reduced structure scale of microdevices increases any effects of surface forces/tension and capillary actions. There is hence a potential to use such forces to deliver and enclose fluid in designed cavities for subsequent applications such as conduction of reactions under changing pressures and temperatures. Although surface tension is able to drive fluid flow without external forces, designing a system that relies completely on capillary forces for the indicated applications is a challenging task.

It has been reported that such a capillary force-driven device enables surface actuated fluid distribution action. The device consists of one or more ‘assay stations’ or ‘wells’, which are located between two distinct multipurpose communication channels. Each of these ‘assay stations’ is connected to both multipurpose communication channels via at least two inlets. A fluid sample enters the first multipurpose communication channel and from there flows into the assay stations. While providing a useful microchip apparatus, a drawback of this device is a potential overflowing of fluid sample from the assay stations into the second multipurpose communication channel. Such overflow will result in the contamination of other assay stations within the respective device.

Another drawback of the above-cited device is the use of displacing liquid in the distribution of the fluid sample. This displacing liquid enters the first multipurpose communication channel, where it displaces the fluid sample. The displacing liquid thus directly contacts the fluid sample. Such contact increases the risk of mixing and hence contamination, in particular where the displacing fluid has not carefully been selected with respect to its properties. In order to remove the fluid sample from the first multipurpose communication channel, it may be required to select a displacing liquid that possesses a high affinity for the surface of the respective channel. However, a liquid with such a high surface affinity may cause the generation of a large capillary force. A large capillary force acting on the first inlet of an assay station may cause the fluid sample to overflow out of the assay station through the second inlet. As a result, the fluid sample may enter the second multipurpose communication channel. From this channel it may get in contact with the fluid sample of other assay stations of the device, thus causing contamination. Furthermore, the process of overflowing may cause a mixing with the displacing liquid, which may affect both the properties of the displacement liquid and a subsequent analysis of the fluid sample in the assay station.

Micro-devices such as the one disclosed by Gong et al. (WO 03/035902) usually require a means to release entrapped air from the sample chamber. Examples of such means, which can be used to release entrapped air, are the application of external force such as centrifugation, pumping, or providing a venting means.

The use of external forces requires either an incorporation of additional peripheral supporting systems such as centrifuges or pumps to the operation process or addition of further functions to the device. These approaches increase the complexity and cost of the device and its operation as well as the overall portability of the process.

Typical uses of the above-described device are the performance of a reaction in its assay stations or storage subsequent to an analysis. Where a respective device that is to be used in one of these ways comprises a vent, the vent needs to be sealed to allow enclosure of the fluid sample. The required sealing process however results in a contact between the fluid sample in the sample chamber and the surface of the respective sealing material. This contact bears the risk of fluid sample flowing out due to a displacement of fluid sample by the sealing material. Fluid sample may thus enter one of the multifunctional channels of the respective device. Similarly to the use of displacing liquid, fluid sample entering a multifunctional channel may contaminate fluid sample in other reaction chambers of the device.

### SUMMARY OF THE INVENTION

In one aspect, the invention thus relates to a microfluidic device for analysing a fluid sample, said device comprising:
at least one sample transmission channel;
at least one multi-functional channel; and
at least one reactor module fluidly connecting the at least one sample transmission channel to the at least one multi-functional channel, said at least one reactor module comprising:
   at least one reaction chamber having at least one inlet in fluid communication with the at least one sample transmission channel, and
   at least one fluid isolation chamber, the fluid isolation chamber being in fluid communication with at least one outlet of the at least one reaction chamber,
      wherein said at least one fluid isolation chamber isolates the fluid sample from the at least one multi-functional channel.

In another aspect, the invention thus relates to a method of detecting an analyte in a fluid sample, comprising:

(a) providing the above-mentioned microfluidic device for detecting an analyte in a fluid sample, comprising:
at least one sample transmission channel;
at least one multi-functional channel; and
at least one reactor module fluidly connecting the at least one sample transmission channel to the at least one multi-functional channel, said at least one reactor module comprising:
   at least one reaction chamber having at least one inlet in fluid communication with the at least one sample transmission channel, and
   at least one fluid isolation chamber, the fluid isolation chamber being in fluid communication with at least one outlet of the reaction chamber, wherein said at least one fluid isolation chamber isolates the fluid sample from the at least one multi-functional channel.
      (b) loading the fluid sample into said device,
      (c) sealing the at least one sample transmission channel and the at least one multi-functional channel with a sealing material, and
      (d) carrying out at least one analyte detection reaction, said reaction providing at least one qualitative or quantitative datum relating to the analyte.

Throughout the description and the claims, the meaning of the terms “analyse”, “analysis” or “analysing” as applied to the fluid sample are not restricted to only their conventional meaning. Accordingly these terms refer to any act that is carried out to quantitatively and/or qualitatively detect (e.g. measure, evaluate or determine) a property or characteristic of the fluid sample. In addition, these terms as used herein refer to any act of distributing or enclosing a fluid sample (e.g. for purposes of observing flow distribution behaviour of a fluid sample within an enclosed space) without carrying out any quantitative and/or qualitative method of detection on the fluid sample. Furthermore, the terms as used herein also refer to the act of storing a fluid sample (e.g. for the purposes of studying the interaction of a fluid sample with a chosen substrate material over a long period of time within an enclosed space).

Reference numbers that accompany terms used in the description to describe any part of the device according to the invention are meant for illustration purposes only, and should not be construed to limit that part of the device to the particular structure/compartment as illustrated and as indicated by the reference numbers in the figures.

### BRIEF DESCRIPTION OF THE DRAWINGS

In order that the present invention may be fully understood and readily put into practical effect, there shall now be described by way of non-limitative example only preferred embodiments of the present invention, the description being with reference to the accompanying illustrative drawings.

In the drawings:

FIG. 1 is a plan view of a first embodiment;

FIG. 2 is a plan view of two embodiments;

FIG. 3 is a plan view of a four embodiments;

FIG. 4 is a plan view of two further embodiments;

FIG. 5 is a side view of another embodiment;

FIG. 6 is side views of two further embodiments;

FIG. 7 is a side view of yet further embodiments;

FIG. 8 is cross-sectional view of the embodiment of FIG. 5;

FIG. 9 is a further cross-sectional view of the embodiment of FIG. 5;

FIG. 10 is a plan view of a further two embodiments;

FIG. 11 illustrates the loading of a fluid sample;

FIG. 12 illustrates the distribution of the fluid sample;

FIG. 13 shows the sealing process;

FIG. 14 shows the completion of the sealing process;

FIG. 15 illustrates three substrate layers; and

FIG. 16 is an illustration of fluorescence emission.

### DETAILED DESCRIPTION OF THE INVENTION

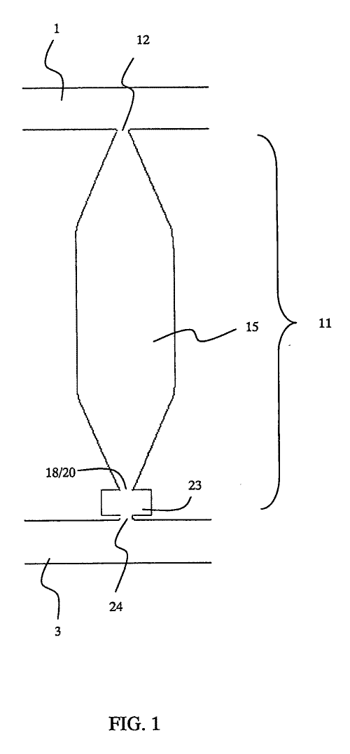

As shown in FIG. 1, the microfluidic device includes at least three compartments, namely, one or more sample transmission channels 1, one or more multi-functional channels 3 and at least one reactor module 11, each of which may include other various sub-compartments (which are in the following for convenience likewise addressed as compartments). The at least one sample transmission channel 1 may be located at any position within the device, as long as its general orientation allows for the conduction of a fluid sample from one or more loading ports 5 of the device to the one or more reactor modules 11. If the sample transmission channel 1 is in fluid communication with more than one loading port 5, the additional loading port(s), such as loading ports 6 or 9 in FIG. 10 may be of the same or different shape and surface characteristics than loading port 5 or than each other. In embodiments with several loading ports 5, 6 etc., some of these loading ports may be dedicated to accommodate a fluid sample from the environment, e.g. a user, while other loading ports may be dedicated to other functions. Such other functions may for instance include serving as a reservoir for an excess of fluid that has been filled into the sample transmission channel via another loading port. The respective loading ports 5, 6 or 9 etc. may be of any depth, as long as its volume does not prevent an isolation medium from performing its function when filled into the loading port after loading with a fluid sample. As an illustrative example, two loading ports 5 and 6 (see e.g. FIG. 10A) may be in fluid communication with a sample transmission channel 1, of which loading port 5 may be dedicated to accommodate both a fluid sample and an isolation media. If loading port 5 is deeper than channel 1, it may retain the fluid sample after loading the device with the same. Subsequently a sealing fluid may be used an isolation media (see below), which may be miscible with the respective fluid sample. When said sealing fluid is disposed into loading port 5, the fluid sample present therein will for instance dilute the isolation media. The depth of the loading port 5 is then limited to the volume at which this dilution does not avert the function of the sealing fluid (see below). The sealing fluid may be of such low viscosity that it immediately also flows through channel 1 and enters loading port 6. In such cases the same requirements as for loading port 5 may also apply for loading port 6. Typically, at least one of the ports in communication with channels 1 or 3 thus provides a small volume, with a depth of less than about 0.5 mm.

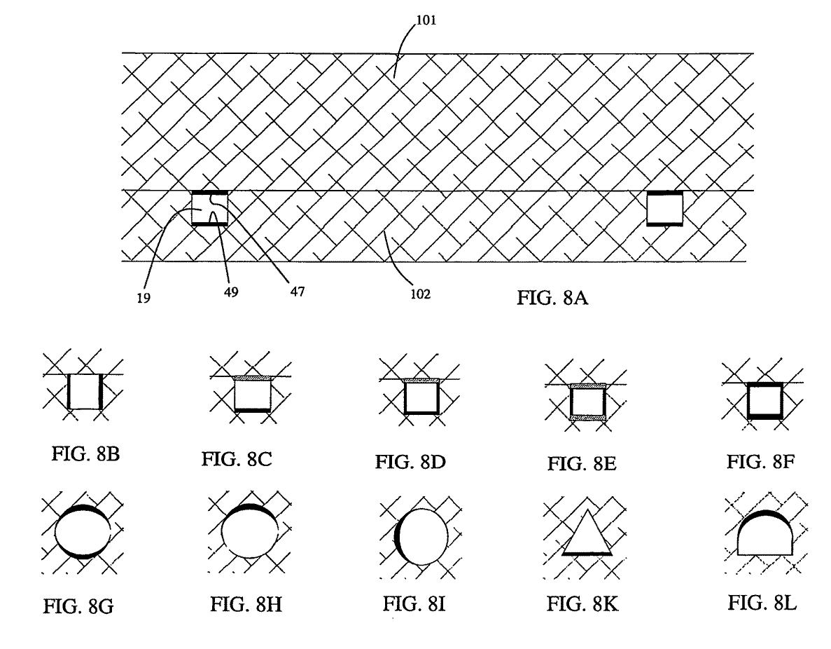

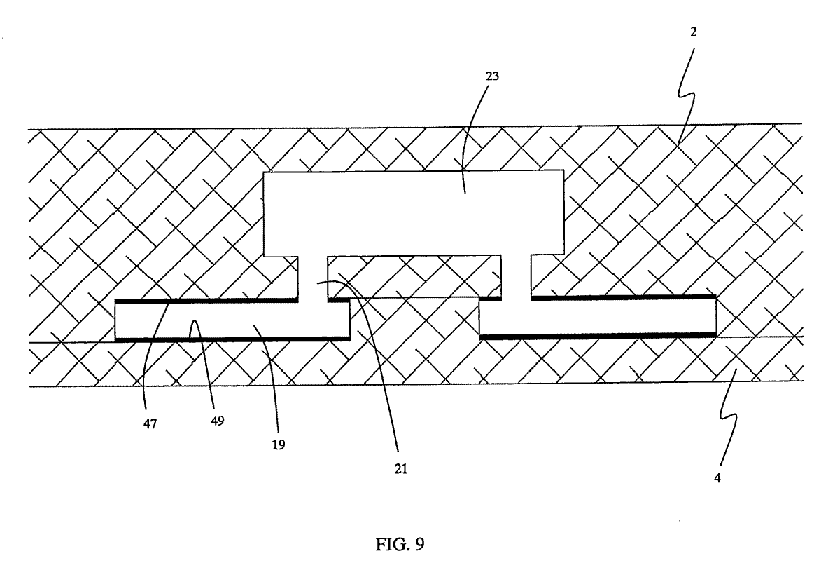

The sample transmission channel(s) 1 may possess any internal surface characteristics, as long as they allow for the conduction of a fluid sample. Where for instance an aqueous fluid sample is provided, internal surfaces of the channels may thus be rendered hydrophilic or hydrophobic. Furthermore, different internal areas of channel(s) 1 may provide different surface characteristics. Thus, some areas on the sample transmission channel(s) 1, such as walls or wall-portions, may be rendered hydrophilic, while others areas may be rendered hydrophobic. FIG. 8 depicts examples of differently treated inner walls of channels of a square, triangular and circular profile. In typical embodiments, the sample transmission channel(s) 1 provide surface characteristics that allow the conduction of a fluid sample to a lesser degree than respective surface characteristics of the reaction chamber(s) 15 of the reactor module(s) 11.

A treatment of the sample transmission channel(s) 1 or any other part of the device that achieves an alteration of surface characteristics may be any treatment that leads to an alteration of the respective surface characteristics that lasts long enough for a subsequent conduction of fluid sample to be affected. Typically, this treatment does not affect the composition of a fluid sample contacting the respective surface area. In some embodiments the treatment does not affect the composition of any fluid that contacts the respective surface area. In other embodiments the treatment may for instance alter an isolation medium if filled into the sample transmission channel(s) 1 (see below).

Treatment that may be carried out to alter surface characteristics may comprise various means, such as mechanical, thermal, electrical or chemical means. A method that is commonly used in the art is a treatment with chemicals having different levels of affinity for the fluid sample. As an example, the surface of plastic materials can be rendered hydrophilic via treatment with dilute hydrochloric acid or dilute nitric acid. As another example, a polydimethylsiloxane (PDMS) surface can be rendered hydrophilic by an oxidation with oxygen or air plasma. Alternatively, the surface properties of any hydrophobic surface can be rendered more hydrophilic by coating with a hydrophilic polymer or by treatment with surfactants. Examples of a chemical surface treatment include, but are not limited to exposure to hexamethyldisilazane, trimethylchlorosilane, dimethyldichlorosilane, propyltrichlorosilane, tetraethoxysilane, glycidoxypropyltrimethoxy silane, 3-aminopropyltriethoxysilane, 2-(3,4-epoxy cyclohexyl)ethyltrimethoxysilane, 3-(2,3-epoxy propoxyl)propyltrimethoxysilane, polydimethylsiloxane (PDMS), γ-(3,4-epoxycyclohexyl)ethyltrimethoxysilane, poly (methyl methacrylate), a polymethacrylate co-polymer, urethane, polyurethane, fluoropolyacrylate, poly(methoxy polyethylene glycol methacrylate), poly(dimethyl acrylamide), poly[N-(2-hydroxypropyl)methacrylamide] (HPMA), α-phosphoryl-choline-o-(N,N-diethyldithiocarbamyl)undecyl oligoDMAAm-oligo-SThlock co-oligomer (see Matsuda, T et al., Biomaterials (2003), 24, 24, 4517-4527), poly(3,4-epoxy-1-butene), 3,4-epoxy-cyclohexylmethylmethacrylate, 2,2-bis[4-(2,3-epoxy pro-poxy)phenyl]propane, 3,4-epoxy-cyclohexylmethylacrylate, (3′,4′-epoxycyclo-hexylmethyl)-3,4-epoxycyclohexyl carboxylate, di-(3,4-epoxycyclohexylmethyl)adipate, bisphenol A (2,2-bis-(p-(2,3-epoxy propoxy)phenyl) propane) or 2,3-epoxy-1-propanol.

Likewise, the sample transmission channel(s) 1 may possess any geometric characteristics, as long as they allow for the conduction of a fluid sample. They may for instance be straight, bend (as for instance in FIG. 10B) or helical, contain loops, as well as contain additional internal geometric characteristics. Such internal geometric characteristics may include, but are not limited to, a change in diameter, inversions, grooves or dents. In some embodiments, the shape of the transmission channel(s) provides geometric characteristics that assist the conduction of a fluid sample. In other embodiments, for instance where several channels of different geometric characteristics are in fluid communication, the shape of the transmission channel(s) provides to a certain lower or higher degree geometric characteristics that assist or retard the conduction of a fluid sample, in particular in relation to respective further transmission channel(s).

The sample transmission channel(s) 1 may be of any length, linear or branched and posses a transverse section of any profile. Examples of respective profiles include, but are not limited to, the shape of a circle, an egg, letters V or U, a triangle, a rectangle, a square, or any oligoedron. Typically, the diameters of the sample transmission channel(s) are selected within the range of about 5 micrometers to about 5 millimeters.

As indicated above, at least one sample transmission channel 1 is in fluid communication with one or more loading ports of the device. This loading port 5—or these loading ports 5, 6 and 9 etc.—may serve in accommodating a fluid sample or isolation-medium. Furthermore, the sample transmission channel(s) 1 are in fluid communication with at least one reaction chamber 15 of at least one reactor module 11. A respective reaction chamber may vertically be located at the same or a different level than the sample transmission channel(s) 1. In embodiments where it is located vertically below the level of the sample transmission channel(s) 1, the difference in elevation may assist the conduction of a fluid sample from the sample transmission channel(s) 1 into the at least one reaction chamber 15.

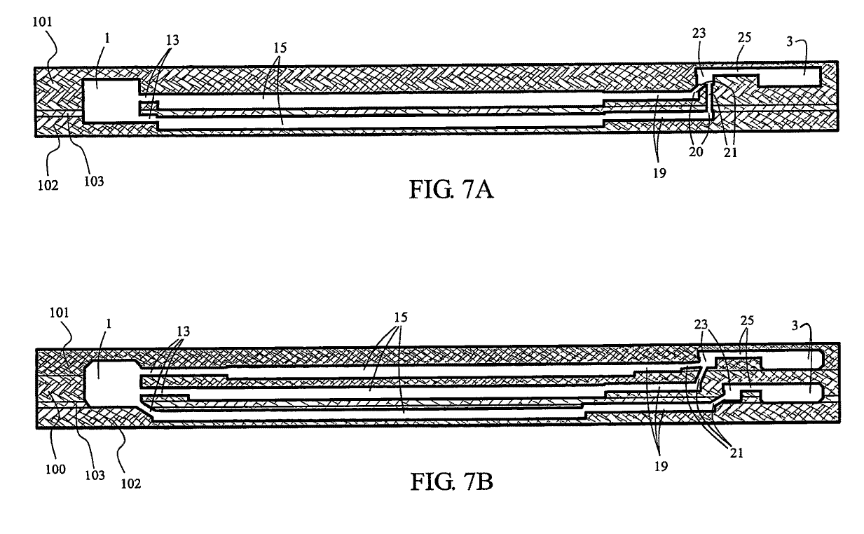

In embodiments where one reactor module 11 contains more than one reaction chamber 15, these chambers may be of identical dimension and located in positions exactly on top of each other. In such embodiments there may be disposed a different reactive compound in each reaction chamber (see below). It may be desired to use such a device for simultaneous analytical measurements, using for instance different wavelengths of irradiation. In other embodiments the respective chambers may be of different dimension and/or located at positions that are horizontally different (see e.g. FIG. 7B). Such embodiments may be desired in order to have control areas, in order to verify that each detection is independent from signals of different chambers of the device.

The terms “horizontal”, “vertical” and “on top” as used herein, refer to a position, where the device of the present invention is held in such a way that at least one reactor module 11, the multi-functional channel(s) 3 and at least one sample transmission channel 1 are oriented sidewise or alongside, i.e. not on top of each other. In some embodiments, this position reflects an orientation of the device, where any openings such as loading ports 4 to 9 are facing upward, and in which the device can be placed onto a flat surface.

Accordingly, in some embodiments the sample transmission channel(s) 1 are in fluid communication with a plurality of reactor modules 11. The plurality of reactor modules may in some embodiments be arranged in such a way that external means or capillary action fill the plurality of reactor modules simultaneously with the fluid sample 31 via at least one sample transmission channel 1 from any of the one or more loading ports 5 and 6 etc. of the device that are in fluid communication with the respective sample transmission channel. In other embodiments the plurality of reactor modules may be arranged in such a way that a sequential filling of these reactor modules occurs. Likewise, if several reaction chambers 15 are provided for within a reactor module 11, these reaction chambers may be arranged in such a way that external means or capillary action fill them simultaneously or sequentially. Furthermore, the plurality of reactor modules may be arranged so as to provide for instance a simultaneous or a sequential filling of sample transmission channels 1 with an isolation medium 33 to physically separate the plurality of reactor modules.

In other embodiments there may be provided a plurality of sample transmission channels 1. As an example, each of such sample transmission channels 1 may be in fluid communication with just one reactor module and one loading port 5, 6 etc. Such embodiments may for example be desired where different fluids, such as buffers, organic solvents or ionic liquids are to be tested with respect to their suitability for a specific reaction.

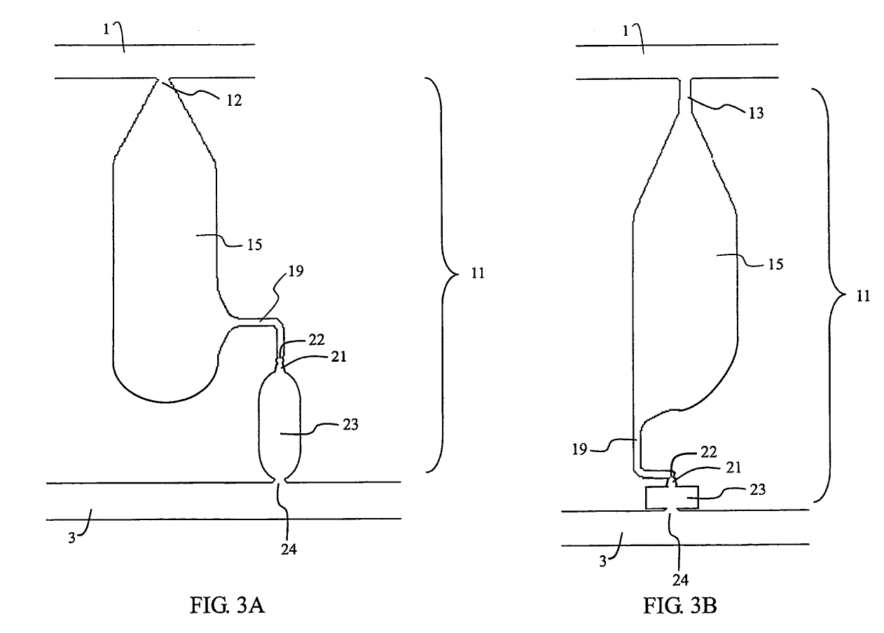

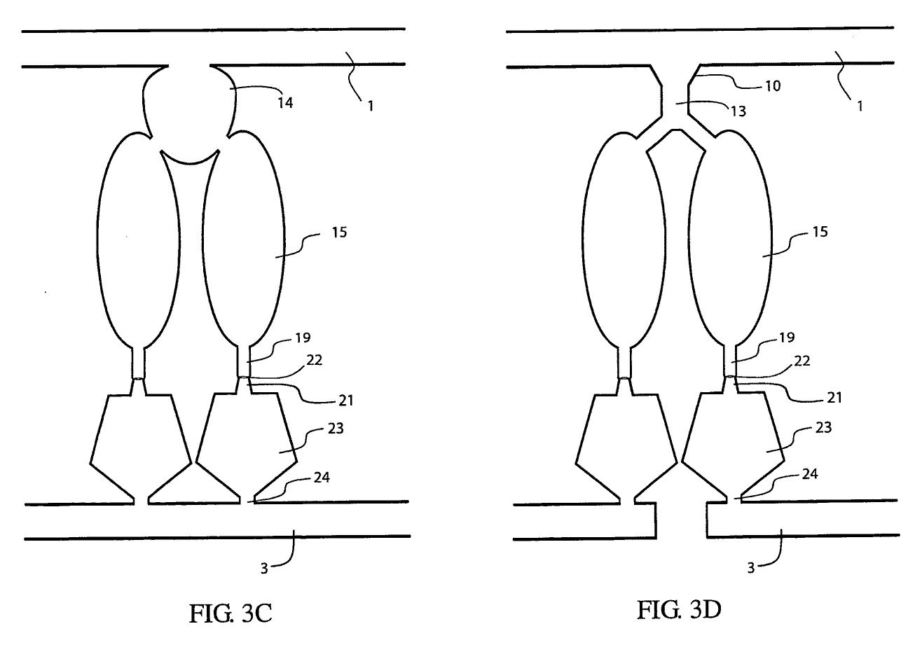

The device of the present invention furthermore comprises at least one multi-functional channel 3. In some embodiments, this channel may consist of one single unit, while in other embodiments it may form several portions, which are not in direct connection with each other (see e.g. FIG. 3D). The multi-functional channel(s) 3 may be of any length, linear or branched (see e.g. FIG. 10B).

The multi-functional channel(s) may be of any surface characteristics. In some embodiments it/they may posses an internal surface area with surface characteristics that retard the conduction of a fluid sample. Where for instance a fluid sample is provided, which is aqueous, an inner surface of a multi-functional channel 3 may be hydrophobic or may be treated in such a way that they provide hydrophobic surface characteristics. In other embodiments the multi-functional channel 3 may posses an internal surface area with internal surface characteristics that assist the conduction of a fluid sample. In such embodiments it may thus resemble the sample transmission channel(s) 1 in this respect.

Likewise, the shape of the multi-functional channel(s) 3 may provide any geometric characteristics, as long as it allows for the accommodation of an isolation-medium and air. In some embodiments, the shape of a multi-functional channel 3 provides geometric characteristics that retard the conduction of a fluid. In other embodiments the shape of a multi-functional channel may posses geometric characteristics that assist the conduction of a fluid. The multi-functional channel(s) 3 may serve in accommodating an isolation-medium such as a sealing material. Such an isolation-medium may be placed and/or flow into the multi-functional channel(s) 3 and subsequently be solidified into a rigid or semi-rigid enclosure surfaces. It should be noted that the at least one sample transmission channel 1 may likewise serve in accommodating an isolation-medium.

The multi-functional channel(s) 3 may be of any length and possesses a transverse section having any suitable profile. Examples of respective profiles include, but are not limited to, the shape of a circle, an egg, letters V or U, a triangle, a rectangle, a square, or any oligoedron. Typically the diameters of the sample transmission channel(s) are selected within the range of about 5 micrometers to about 5 millimeters.

The one or more multi-functional channel 3 is in fluid communication with one or more loading ports 4, 7, and 8 etc. (see e.g. FIG. 10B). These loading ports are able to accommodate air or an isolation-medium and allow for its transfer to the multi-functional channel 3. The potentially various respective loading ports 4, 7, and 8 etc. may be of the same or of different shape and surface characteristics. They may furthermore posses the same or different shape and surface characteristics as the loading ports 5, 6 and 9 etc, which are in fluid communication with the sample transmission channel(s) 1. Furthermore, where the multi-functional channel 3 is in communication with more than one loading port 7, the additional loading port(s), such as loading port 8 in FIGS. 10A and B may be of the same or different shape and surface characteristics than loading port 7 or than each other.

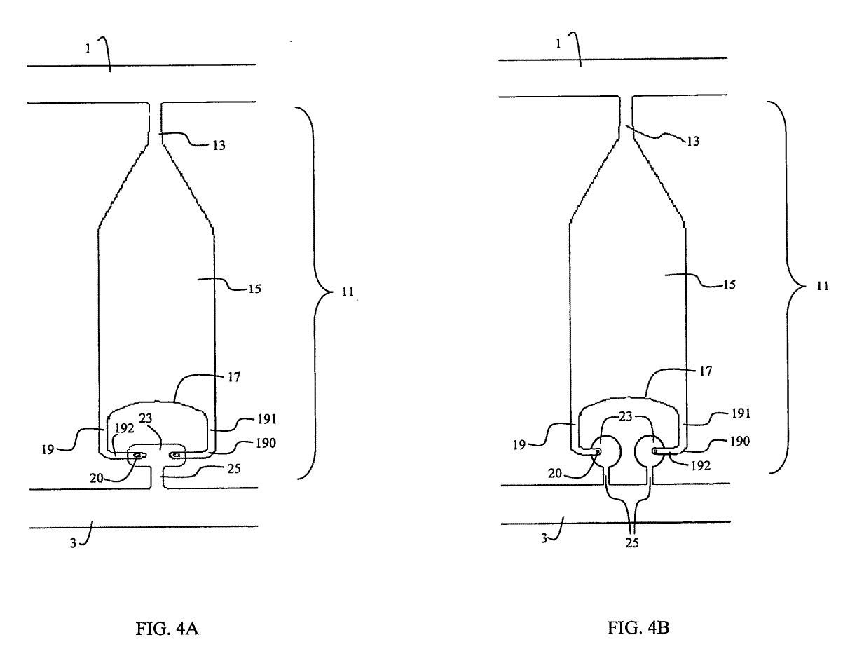

Additionally, the multi-functional channel 3 is in fluid communication with the fluid isolation chamber(s) 23 of each of the one or more reactor modules. In typical embodiments of the device of the invention, such communication is provided for by an outlet 24. This outlet may be of any form that provides a connection between the multi-functional channel 3 and the fluid isolation chamber(s) 23. Examples of outlets 24 include, but are not limited to, openings, valves, necks or channels. FIG. 4 illustrates two exemplary embodiments, where the outlet takes the form of a channel 25. Such a channel may take any suitable form of any length that provides a fluid communication to the fluid isolation chamber 23, for instance straight linear, spirally twisted or bended to any degree. It may furthermore contain additional internal geometric characteristics such as for example a change in diameter, inversions, dents or grooves. It may possess an internal surface area of any surface characteristics, as long as it does not prevent the communication of air between the reactor module 11 and the multi-functional channel 3. It should be noted that an outlet 24, such as for instance in form of a channel 25, may permit the entry of liquid into the fluid isolation chamber(s) 23. If desired, its geometric and surface characteristics may however also be selected to prevent such entry of liquid.

The cross section of channel 25 may be of any shape, as long as it does not prevent the conduction of a fluid such as air or a fluid sealing material. Examples of respective profiles include, but are not limited to, the shape of a circle, triangle, rectangle, square, or any oligoedron. Typically, the diameter of channel 25 is about the same or smaller than at least one diameter of the respective multi-functional channel 3. As an example, where a multi-functional channel 3 has a vertical diameter of 0.2 millimetres and a horizontal diameter of 0.65 millimetres, a diameter of the microcapillary channel(s) 19 is typically selected in the range of about 5 micrometers to about 0.65 millimetres. It may then for instance take a vertical diameter of 0.1 millimeters and a horizontal diameter of 0.15 millimeters.

The opening of the respective outlet may be of any shape. Examples of respective profiles include, but are not limited to, the shape of a circle, triangle, rectangle, square, or any oligoedron. In embodiments where the outlet 24 takes the form of a channel 25, the opening may have similar dimensions as the profile of channel 25. In other embodiments providing a channel 25, a wall may separate the channel 25 from the respective multi-functional channel 3. Such a wall may contain one or more openings of smaller dimensions and thus allows for a fluid communication with the multi-functional channel 3.

In the absence of other fluid such as a fluid sample, the air in the multi-functional channel(s) 3 is therefore in contact with the air in the reactor module(s). This is in turn is in contact with the air in the sample transmission channel(s) 1, thus forming one integrated air-filled system. As a consequence, during the filling of the sample transmission channels 1 and the reactor modules 11 with a fluid sample, the multi-functional channel(s) 3 generally act(s) as a vent to allow for the release of entrapped air. However, where a multi-functional channel 3 is filled with an isolation-medium, it will not function as a vent anymore. Instead it will seal the reactor modules. Hence, no fluid is able to enter the reactor module(s) 11 via the outlet 24 of the fluid isolation chamber(s). Reactor module(s) 11 are thus isolated from air that is in contact with the one or more loading ports that are connected to the multi-functional channel(s) 3. They are also isolated from any liquid which may get in contact with the respective loading ports.

As indicated above, the device of the present invention may provide a plurality of reactor modules. In some embodiments the reactor modules 11 may thus be arrayed in high density, either in two-dimensions or in three-dimensions, with each reactor module comprising one or several reaction chambers 15. The respective reactor modules may be in communication with any number of the same or different sample transmission channels 1.

Typically, these reaction chambers 15 provide internal surface characteristics that assist the conduction of a fluid sample to the same or to a higher degree than at least one of the sample transmission channels 1 that are in fluid communication with it. In some embodiments, it may be desired to provide multiple reaction chambers 15 with different internal surface characteristics. Thus, some reaction chambers, whether within the same or among different reactor modules, may provide internal surface characteristics that assist the conduction of a fluid sample to a different degree than those of other reaction chambers.

In some embodiments it may furthermore be desired to provide reaction chamber(s) 15 that provide internal surface characteristics, which assist the conduction of a fluid sample to a higher degree than all sample transmission channels 1 that are in fluid communication with it. Such embodiments assist a flow of a fluid sample 31, driven by capillary forces or external means, from the loading port(s) 5, 6 and 9 etc. of the device that are in fluid communication with the sample transmission channels 1 to the reaction chamber 15 of a reactor module 11. Some embodiments are thus able to completely rely on capillary forces to achieve a filling of for instance all reaction chambers in all reactor modules 11 on the device of the present invention. In other embodiments, where it may be desired to provide a plurality of sample transmission channels of various internal surface characteristics, it may be required to use some force in order to fill all sample transmission channels and all compartments of the reactor modules. Such force may for instance be provided by a gentle pressing of fluid with a pipette into a loading port, which is in fluid communication with a sample transmission channel 1, e.g. loading ports 5, 6 or 9 in FIG. 10B.

The reaction chamber(s) 15 may be of any shape, as long as the desired reaction can be performed within the reaction chamber(s). In typical embodiments the reaction chamber(s) 15 will be of a shape that allows for a complete filling with a fluid sample. Examples of such shapes include, but are not limited to rectangle, square, ovoid, circular and bottle-like shapes. Optionally, a shape of the reaction chamber(s) 15 may be selected that avoids or prevents the formation of air bubbles during the process of filling with fluid sample 31. Examples of means to avoid the formation of air bubbles include, but are not limited to, straight or convex walls or wall portions and rounded corners.

In typical embodiments, the reaction chamber(s) 15 have a volume ranging from about 1 pico liter to about 1 milli liters. The volume may thus for instance be selected to be about 100 micro liters or within the range of 500 nano liters to 10 micro liters. The reaction chamber(s) extend in typical embodiments vertically to a distance of the range of 5 micrometers about 5 millimeters. In embodiments where the device of the present invention provides a plurality of reactor modules 11, these reactor modules may be of substantially identical dimensions.

The reaction chamber(s) 15 have at least one inlet 12 and at least one outlet 18. These inlets and outlets may be of any form, thus for instance forming an entrance connection joint. Examples of such inlets and outlets include, but are not limited to, openings, valves, chambers, necks or channels. Where a channel is provided, for instance an inlet channel 13, such channel may also be branched (see e.g. FIG. 3D). Furthermore, such a channel may provide bevelled portions 10 (see e.g. FIG. 3D). In embodiments with more than one reaction chamber, the respective reaction chamber may be connected in parallel and or perpendicular with the sample transmission channel 1 and the respective multi-functional channel 3. The respective inlets and outlets of each reaction chamber may thus differ in their geometrical and surface properties. In embodiments where they provide for instance valves, necks or channels, they may thus also be orientated in different angles relative to each other.

Through one or more of such inlet(s) 12, at least one reaction chamber 15 of each reactor module is fluidly connected to the sample transmission channel(s) 1. In embodiments where inlet 12 provides for instance a neck, a channel 13 or a chamber 14 (see e.g. channel 13 in FIGS. 3B, 4 and 5, and chamber 14 in FIG. 3C), it possesses an internal surface area with internal surface characteristics that allows for the conduction of a fluid sample into the respective reactor module 11. These surface characteristics may thus be identical to those of the sample transmission channel 1 or differ from them. Where an aqueous fluid sample is provided, for instance, a respective inlet may thus be either hydrophilic or hydrophobic. It may also be surface treated in such a way that they provide respective hydrophilic or hydrophobic surface characteristics (see above). In some embodiments, for instance where several reactor modules 11 or where several reaction chambers 15 of a reactor module are connected to the sample transmission channel in parallel, each inlet may provide surface characteristics that assist the flow of fluid sample 31 to a different degree, when compared to each other.

In typical embodiments the inlet(s) 12 provide surface characteristics that assist the conduction of a fluid sample to a comparable or to a greater degree than respective surface characteristics of the respective sample transmission channel 1. Where the sample transmission channel(s) for instance provide partly hydrophilic surface characteristics, the inlet(s) 12 of the reaction chamber 15 or the respective channel(s) 13 may provide comparable or hydrophilic surface characteristics.

In typical embodiments, the inlet(s) 12 or the respective channel(s) 13 or chamber(s) 14 furthermore provide surface characteristics that assist the conduction of a fluid sample to a lesser degree than respective surface characteristics of the respective reaction chamber 15. Where the reaction chamber for instance provides hydrophilic surface characteristics, the inlet(s) 12 or the respective channel(s) 13 may provide less hydrophilic surface characteristics.

Typical embodiments of the device of the present invention thus provide compartments with coordinated surface characteristics. A respective coordination comprises reaction chambers 15 with surface characteristics that assist the flow of a fluid sample, sample transmission channel(s) 1 that assist the flow of a fluid sample to a lesser degree and reaction chamber channel-inlet(s) 13 that assist the flow of a fluid sample to the same or a higher degree than the sample transmission channel(s) 1. Such coordination further assists the overall flow of fluid sample 31 from loading ports(s) 5 and 6 etc. of the device that are in fluid communication with the sample transmission channels through one or more inlets into the reaction chamber 15 of a reactor module 11. Such a coordination furthermore provides for a complete flow of a fluid sample into the reaction chambers of the device, provided that the correct amount matching the volume of all reaction chambers of the device is filled into a respective loading port 5, 6 or 9 etc. A respective coordination thus allows for an arrangement of a device that is able to provide empty sample transmission channels, even where the reaction chambers are filled with a fluid sample.

It should be noted that additional means of the device, means, or a combination thereof may be able to achieve a similar flow of a fluid sample, for instance where it is desired to deviate from the above described coordination of surface characteristics. Means of the device include, but are not limited to, valves and switches, which are well known to the person skilled in the art. A combination of internal and external means include, but are not limited to, electrokinetic methods of flow control or the use of so called “microactuators”. Electrokinetic methods typically comprise the use of integrated electrodes and an applied electric field (see e.g. Schafsfoort, R B M et al., Science, (1999) 286, 942-945). Microactuators are polymer electrolytes or conjugated polymers, which undergo volume changes in an electrical field or during oxidation and reduction (see e.g. Jager, E W H et al., Science, (2000) 290, 1540-1545).

The shape of the reaction chamber inlet(s) 12 or the respective channel(s) 13/chamber(s) 14 may furthermore provide geometric characteristics that further control the flow of a fluid sample. In some embodiments, for instance where several sample transmission channels are in fluid communication with one reaction chamber 15, the shape of each such inlet may, in relation to another inlet, provide to a certain lower or higher degree geometric characteristics that assist or retard the conduction of a fluid sample.

In addition to any optional surface coating that alters its surface characteristics, the reaction chamber(s) 15 may have disposed therein one or more compounds. These one or more compounds may be comprised in a coating to at least one wall or wall portion of the reaction chamber. They may also be deposited as for instance a fluid or solid reactant, reactant solution or dried reactant solution. They may serve as reagents in carrying out an assay reaction to analyse a property of a fluid sample. In embodiments where it is desired to use the device of the invention to perform PCR, the chemical compound may for instance be a primer or a probe. The one or more compounds may also be coupled to reactive groups of a coating such as PHPMA (see above, cf. Carlisle, R C et al., The Journal of Gene Medicine (2004), 6, 3, 337-344) or to an otherwise chemically modified surface portion of the reaction chamber. For example, where the surface is made of PDMS, this polymer may be derivatised with 3-aminopropyldimethylethoxysilane to create reactive amino groups (Blank, K et al., Proc. Natl. Acad. Sci. USA. (2003), 100, 20, 11356-11360).

For some embodiments of the invention, compounds may be used in form of a library. Examples of such libraries are collections of various small organic molecules, chemically synthesized as model compounds, or nucleic acid molecules containing a large number of sequence variants. As an example, each compound of such a library may be disposed into one reaction module of one or more devices. Such compounds may be disposed (before or after the assembly of the devices) in an automated way by commercially available machines, which are well known to those skilled in the art.

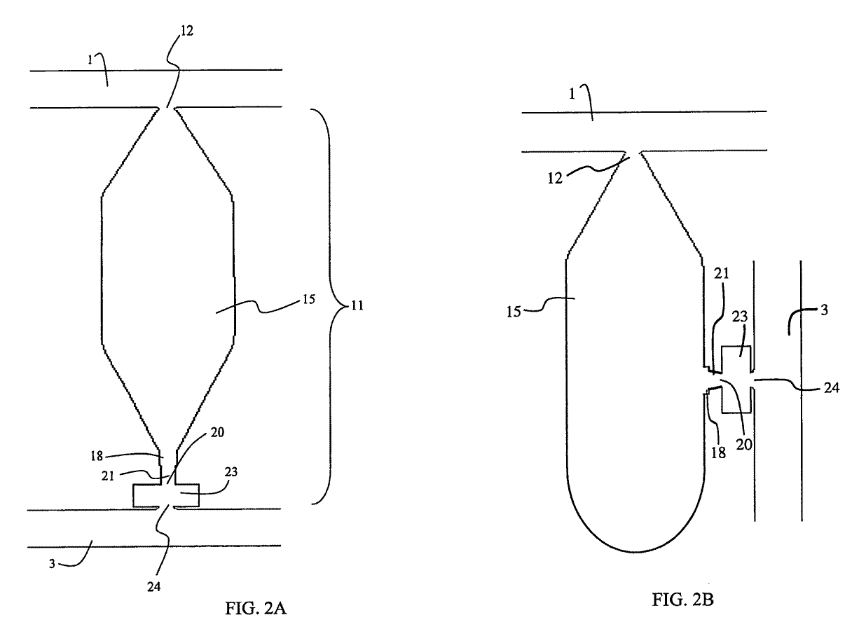

The reaction chamber(s) 15 are in fluid communication with the fluid isolation chamber(s) 23 of the same reactor module 11 via at least one outlet 18. This outlet may be placed at any location relative to the inlet(s) 12 of the reaction chamber. Since no flow through the respective inlet(s) 12 and outlet(s) 18 occurs during sample analysis (see below for the function of chamber 23 in this respect), their relative locations do not affect the function of the device. In some embodiments the outlet(s) 18 may thus for instance point sideward relative to inlet(s) 12 (see e.g. FIG. 2B or 3A). In other embodiments it/they may be located at a distal portion of the reaction chamber 15 with respect to the inlet(s) 13 that provide fluid communication to the sample transmission channel(s) 1. In such embodiments inlet(s) 12 and outlet(s) 18 may thus be located at opposing portions/walls of the reaction chamber, and for instance face each other.

The fluid isolation chamber(s) 23 are on the other hand in fluid communication with the respective reaction chamber via an inlet 20. Examples of such inlets include, but are not limited to, openings, valves, necks or channels. In embodiments, where this inlet 20 takes the form of for instance a channel, it may provide additional surface characteristics or geometric characteristics that retard the conduction of a fluid sample. In embodiments that where a physical distance to the respective reaction chamber 15 is already provided for (see below), the inlet 20 typically takes the form of an opening or a channel with a small length in the direction, which is perpendicular to the surface in which the inlet is formed.

The fluid isolation chamber(s) 23 are in turn in fluid communication with the multi-functional channel(s) 3 via an outlet 24 (see above). The flow of fluid through the outlet(s) 18 of the reaction chamber(s) into the multi-functional channel(s) 3 is thus prevented by the fluid isolation chamber(s) 23. The fluid isolation chamber(s) 23, which are fluidly connected to the reaction chamber outlet(s) 18 and the multi-functional channel(s) 3, therefore serve in controlling potential flow of fluid sample between the outlet(s) 18 and the multi-functional channel(s) 3. The fluid isolation chamber(s) 23 may be of any form, as long as they allows for a communication of air between the reaction chamber(s) 15 and the multi-functional channel(s) 3. Examples of shapes, which a cross-sectional profile of a respective form may take, include, but are not limited to the shape of a circle, ovoid, triangle, rectangle, square, any oligoedron (cf. e.g. FIG. 3C), and bottle-like shapes. The fluid isolation chamber(s) 23 may have differential surface conditions, frictions and/or affinity to the fluid sample 31 at the inlet 20.

They may for instance posses an internal surface portion with internal surface characteristics that retard the conduction of a fluid sample. Where for instance an aqueous fluid sample is provided, an internal surface portion may be either hydrophobic or treated in such a way that it provides hydrophobic surface characteristics. In other embodiments a fluid isolation chamber 23 may assist the conduction of a fluid sample, but for instance less so than the reaction chamber 15. In case of an aqueous fluid sample being provided, an internal surface portion of a fluid isolation chamber 23 may for instance provide surface characteristics, which are hydrophilic, but less so than the reaction chamber. Likewise, the fluid isolation chamber(s) 23 or a part of them may for example possess geometric characteristics that retard the conduction of a fluid sample. It may in other embodiments possess geometric characteristics that assist the conduction of a fluid sample, but for instance less so than the reaction chamber(s) 15. The selection of such coordinated geometric and/or surface characteristics may be desired for embodiments, where the process of analysing a fluid sample is accompanied with conditions that lead to an expansion of the fluid sample present in the reaction chambers 15. Such conditions may for instance comprise a change in temperature.

The fluid isolation chamber(s) 23 serve in providing a resistance to forces, which arise within the device. As an example, in the absence of a fluid isolation chamber such forces may lead to the flow of a fluid sample into a multi-functional channel 3. While the arrangement of compartments of the device already prevents the flow of the fluid sample from the reaction chamber(s) 15 into the multi-functional channel(s) 3, it may be desired to provide additional safety measures in this respect. In some embodiments of the device of the present invention a fluid isolation chamber 23 may thus be selected to be of a volume, which provides storage space for any potential overflow of fluid sample 31 from the reaction chamber 15. Such storage space consequently prevents any flow of fluid sample into the multi-functional channel(s) 3.

In typical embodiments, a fluid isolation chamber 23 is selected to be of a volume which is comparable or lower than the volume of the reaction chamber(s) 15. It may therefore have a volume ranging from about 1 pico liter to about 100 micro liters. Likewise, its horizontal and vertical extensions are typically selected to be of comparable or lower values than at least one respective dimension of the reaction chamber(s) 15. Where for instance a reaction chamber 15 has a maximal horizontal diameter of 1.4 millimeters and a maximal vertical diameter of 0.2 millimeters, diameters of a respective fluid isolation chamber 23 are typically selected to be about 1.4 millimeters or below. An embodiment of a respective fluid isolation chamber 23 may, for instance, take a maximal horizontal diameter of 0.7 millimeters and a maximal vertical diameter of 0.1 millimeters. Likewise, the length of a fluid isolation chamber 23 is typically identical or lower than the length of the reaction chamber 15. The one or more fluid isolation chamber 23 and its transverse section may furthermore be of any shape. Examples of shapes of respective profiles include, but are not limited to, a circle, an egg, the letters U or V, a triangle, a rectangle, a square, or any oligoedron.

As already indicated above, the fluid isolation chamber(s) 23 serve in providing a resistance to forces, which arise within the device. As another example, forces arising within the device may—in the absence of a fluid isolation chamber—lead to the contact of fluid sample 31 at the outlet 18 of the respective reaction chamber 15 with any isolation medium 35, which may have been added into the multi-functional channel(s) 3. Therefore, in another aspect, the fluid isolation chamber(s) 23 generally provide a space that is able to prevent contact between fluid sample 31 in the respective reactor module and any isolation medium in the multi-functional channel(s) 3. As explained above, once fluid sample 31 has got in contact with isolation medium 35, this surface contact may lead to a surface action that causes a flow of a fluid sample within the multi-functional channel(s) 3. During such flow, the fluid sample may get in contact with the fluid sample of other reactor modules. Hence, the fluid isolation chamber(s) 23 also prevent potential contaminations of other reactor modules 11 with fluid sample 31.

In yet another aspect, providing a resistance to forces, the fluid isolation chamber(s) 23 further provide a space for matter expansion. Forces arising within the device may be caused by external forces, such as changes in temperature or pressure. These external forces may in turn lead to internal changes, such as changes in pressure or volume. As an example, a change in temperature may cause an expansion of for instance air or fluid present in the reaction chamber 15, which is in fluid communication with the fluid isolation chamber(s) 23. Such changes typically occur for example during a reaction process performed in the reaction chamber 15, during an enclosure process or any subsequent storage. A person skilled in the art will be familiar with the example of a polymerase chain reaction (PCR) as a part of a fluid sample analysis (see also below). During PCR three different reaction steps need to be repeatedly performed, namely melting double-stranded DNA, binding specific primers, and enzymatically extending these primers. Every switch from one step to the next one typically includes a temperature change. The resulting matter expansion may be of particular relevance, where several reaction chambers are connected within one reactor module.

In this aspect, the fluid isolation chamber(s) 23 may for instance provide a pressure regulator during a change of aggregation state of an isolation medium 33 or 35. As indicated above, such isolation medium may be placed and/or flow into the sample transmission channel(s) 1 and/or the multi-functional channel(s) 3 or parts thereof. The two respective isolation media 33 and 35 may be identical or different. They may provide enclosure surfaces of rigid or semi-rigid nature. A typical example of such an isolation medium is a sealing material in form of a fluid. Examples of such sealing materials include, but are not limited to, gels or liquids.

A sealing material may comprise a polymer that is derived from a photosensitive and/or heat-sensitive polymer precursor. Thus, the sealing material may be formed from a respective precursor after filling into the sample transmission channel(s) 1 and/or the multi-functional channel(s) 3, by polymerisation. Alternatively, an isolation medium may—once filled into the respective channels—be able to change its aggregation state, for instance by curing. Finally, a respective isolation medium may also be of a solid state, but of such a nature that it is activated mechanically, electrically, and/or magnetically. In embodiments, where the isolation medium is a sealing material in form of a polymer, it may upon such activation change its aggregation state, so that it can be filled into the respective channels. Upon polymerisation, curing or “deactivation” (i.e. the reverse of “activation” carried out on the isolation medium) of a respective material, the fluid in the respective channels solidifies, thus providing rigid or semi-rigid enclosure surfaces. Currently used sealing materials include, but are not limited to, polydimethylsiloxane (PDMS) and “Room Temperature Vulcanizing” (RTV) silicon.

Commercially available sealing materials are often colourless, for instance RTV silicon and PDMS are transparent elastomers. In typical embodiments of the present invention the sealing material used is however mixed with at least one visually active pigment. This pigment serves as an aid to visualisation, for example, to differentiate the reaction chamber(s) 15 from the sample transmission channel(s) 1 and the multi-functional channel(s) 3. In particular, the visually active pigment helps to improve visual differentiation between the sealing material and the substrate from which the device is formed, so that the flow of the sealing material through sample transmission channels and through multifunctional channels may be clearly observed. Examples of visually active pigments include, but are not limited to, carbon pigments, organic dyes and fluorescent dyes.

Such differentiation may for instance be desired during sealing in order to monitor the sealing process. Such differentiation may also be desired during the measurement of a reaction in the reaction chamber(s) 15. During such measurements this differentiation can be carried out, because at this stage the respective channels are filled with sealing material 33 and 35 (see below).

Additionally, a person skilled in the art will be aware of the fact that a sealing process may be of reversible or irreversible nature. As an example, without oxidative treatment PDMS forms a non-covalent reversible seal with smooth surfaces. In some embodiments it may be desired to reuse a fluid sample contained in the reactor module(s) 11 of a respective device of the invention. In such cases it may be desirable to use a reversible sealing. An irreversible sealing of PDMS contacting for instance glass, silicon, polystyrene, polyethylene or silicon nitride can be achieved by an exposure to an air or oxygen plasma.

It should furthermore be noted that alternative and/or additional sealing means may be used or be part of the device (see below). Examples of such alternative means are a respective substrate layer of the device with for instance self-closing properties, or lids or tapes on any part of the device, for instance loading ports 4 to 9.

As indicated above, the fluid isolation chamber(s) 23 serve in providing a resistance to forces, which arise within the device. Where an isolation medium performs the function of sealing channel 1 or channel 3 as just elaborated, the respective process may give rise to such forces. As an example, the solidification process of an isolation medium may for instance involve or require temperature, pressure and/or volume changes. The solidification process may also lead to a reaction involving changes in temperature, pressure and/or volume. It should be noted that such changes occurring in the sample transmission channel 1 will be communicated via the reactor module 11 to the outlet 18 of the reaction chamber(s), which are in fluid communication with the fluid isolation chamber(s) 23. The latter chamber(s) 23 may therefore serve as a general pressure regulator within the device of the present invention.

In some embodiments a physical distance between the inlet 20 and the outlet 24 of a fluid isolation chamber contributes furthermore to the function of the fluid isolation chamber(s) 23. The conjunctions may for instance be located on opposing surfaces of a fluid isolation chamber. Inlet and outlet may thus in such embodiments face each other.

In some embodiments there may furthermore be provided for a physical separation of a fluid isolation chamber 23 and the respective reaction chamber 15, which is in fluid communication with it. Such separation may be selected in such a way that the fluid communication between the outlet 18 of the respective reaction chamber and the inlet 20 of the fluid isolation chamber is achieved via additional, interconnected means. Such additional means may preferably be designed in such a way that the fluid isolation chamber 23 and the respective reaction chamber 15 are vertically on a different level or vertically separated. Furthermore the fluid isolation chamber 23 may be vertically on a different level from both the respective reaction chamber 15 and the respective multi-functional channel 3. Thus, both the respective reaction chamber 15 and the multi-functional channel 3 may for instance be at a comparable vertical level, while the fluid isolation chamber 23 is located above or below them. In such embodiments any fluid in the multi-functional channel 3 would theoretically have to flow upwards either into the fluid isolation chamber 23 or into the respective reaction chamber 15, if it was to contaminate the reaction chamber. Due to the capillary forces within the microdevice, such upward flow can practically be prevented by means of respective geometrical or surface characteristics, as explained below.

Hence, reaction chamber(s) 15, fluid isolation chamber(s) 23, and multi-functional channel(s) 3 may be located at several different levels within the device. In embodiments where a reactor module contains one reaction chamber 15, one fluid isolation chamber 23, and one multi-functional channel 3, these three compartments may thus be located on three different levels. In embodiments where a reactor module contains three reaction chambers 15, two fluid isolation chambers 23, and two multi-functional channel 3, these seven compartments may thus be located on up to seven different levels (see e.g. FIG. 7 for an illustration). As explained above, a vertical physical separation of chambers 15 and 23, and a multi-functional channel 3 may contribute to the function of a fluid isolation chamber 23. Furthermore, such embodiments provide an additional safety measure in that they prevent any potential contact between a fluid sample in the reaction chamber and any material present in the fluid isolation chamber 23. Should any material enter the fluid isolation chamber from the multi-functional channel(s) 3, as for instance isolation medium, it is still isolated from the reaction chamber due to the physical separation. In other embodiments, such physical separation may also prevent fluid sample 31 from flowing from the reaction chamber 15 into the fluid isolation chamber 23, regardless of the presence of differential surface conditions, frictions and fluid sample affinity.

An example of a physical separation of the outlet 18 of a respective reaction chamber and the inlet 20 of a fluid isolation chamber is the presence of an additional fluid control element between the reaction chamber 15 and the fluid isolation chamber 23. In some embodiments, such a fluid control element may be an inclined port 21. In embodiments, where the fluid isolation chamber 23 and the respective reaction chamber 15 are located on vertically different levels, such a port is thus typically inclined. The angle formed between the base of the fluid isolation chamber 23 and a lateral wall of such a port 21 may thus be of any value in the range between 0° and 180°. In preferred embodiments this angle is selected in the range between about 45° and about 135°, in most preferred embodiments lateral wall of such a port is perpendicular to the base of the fluid isolation chamber 23. For embodiments where port 21 is directly connected to the fluid isolation chamber 23, it should be noted that port 21 may enter any portion of the fluid isolation chamber 23. Examples of such a portion are base walls, top walls or side walls of the fluid isolation chamber.

The port 21 may be of any form that allows for a fluid communication with the fluid isolation chamber 23. Examples of a port include, but are not limited to, a channel, a neck, a chamber or a valve. A cross section of the port 21 may be of any suitable profile. Examples of respective profiles include, but are not limited to, the shape of a circle, ovoid, a triangle, a rectangle, a square, or any oligoedron. In embodiments, where the port 21 is a channel, the maximal size of such a channel in terms of its width is typically of the same or smaller dimensions as the respective cross section of a fluid isolation chamber 23 into which it enters. As an example, a port of circular profile may enter a wall (whether horizontal, vertical or inclined) of a fluid isolation chamber, which may be of circular profile at right angle to the level at which the port enters the chamber 23. The diameter of the respective profile of the fluid isolation chamber may be 0.1 millimeters. In this case the maximal diameter of the respective channel is typically selected to be about 0.1 millimeters or below. It may for instance have a value of 0.05 millimeters.

The port 21 may posses any surface and geometrical characteristics, as long as it allows for the communication of air between the reaction chamber 15 and the fluid isolation chamber 23. It may thus have one or more internal surface portions with internal surface characteristics that retard, prevent or assist the conduction of a fluid sample.

As indicated above, the outlet 18 of the reaction chamber 15 may have the form of for instance an opening, a valve or a channel. In a currently preferred embodiment, it is a microcapillary channel 19. Typically, the reaction chamber will thus provide at least one microcapillary channel, which provides fluid communication with the fluid isolation chamber(s) 23. Such microcapillary channel thus possesses an opening 22 for a fluid communication with a fluid isolation chamber. It thus for instance connects it to an inclined port 21, as illustrated in FIG. 3. The size of the corresponding opening 22 in terms of its width (e.g. its diameter) is smaller than the respective size of the microcapillary channel 19 itself. Respective cross-sectional sizes may differ from about 1.5-fold to about 20-fold, more preferably from about 2- to about 10-fold, and most preferably from about 3- to about 6-fold. Furthermore, the opening 22 is typically smaller than the respective size with respect to the width (e.g. the diameter) of a port 21, if present in the respective embodiment of the device. The opening 22 may furthermore be of any shape. Examples of respective shapes include, but are not limited to, a circle, an egg, letters V or U, a triangle, a rectangle, a square, or any oligoedron. As an example, a suitable circular opening of a microcapillary channel 19 of circular profile with a diameter of 0.1 millimeters may thus be selected to have dimensions of 0.05×0.07 millimeters.

The microcapillary channel(s) 19 may have any suitable form of any length that provides a fluid communication to the fluid isolation chamber 23, for instance straight linear (cf. e.g. FIG. 3C), spirally twisted or bended to any degree (e.g. FIGS. 3A and 3B) or contain loops. They may furthermore be branched, for instance in order to provide communication with two different fluid isolation chambers. The microcapillary channel(s) 19 possess one or more internal surface areas, which provide internal surface characteristics that retard the conduction of a fluid sample. Where for instance an aqueous fluid sample is provided, the inner surface of the microcapillary channel(s) 19 may be either hydrophobic or treated in such a way that it provides hydrophobic surface characteristics (see e.g. FIG. 8). In some embodiments, the shape of the microcapillary channel(s) 19 provides geometric characteristics that further retard the conduction of a fluid sample. Such internal geometric characteristics may include, but are not limited to, a change in diameter, inversions, grooves or dents. The microcapillary channel(s) 19 therefore assist the function of the fluid isolation chamber(s) 23 in preventing the flow of fluid from the reactor module 11 into the multi-functional channel(s) 3. The transverse section of the microcapillary channel(s) 19 may be of any suitable profile. Examples of respective profiles include, but are not limited to, the shape of a circle, an egg, letters V or U, a triangle, a rectangle, a square, or any oligoedron (cf. FIG. 8 for examples). Typically, the size in terms of the width of the microcapillary channel(s) 19 is about the same or smaller than the vertical extension of the respective cross section of the reaction chamber. As an example, where the reaction chamber 15 has a maximal vertical extension of 0.2 millimeters, the maximal diameter of a respective microcapillary channel 19 of ovoid profile is typically selected in the range of about 5 micrometers to about 0.2 millimeters, for example at about 0.1 millimeters.

In some embodiments the components of the reactor module(s) 11 and sample transmission channel(s) 1 are arranged in such a way that—upon filling of fluid sample 31 into the inlets 5 and 6 etc.—capillary action fills the reactor module(s) 11 up to the end of the outlet(s) of the respective reaction chambers. Hence, the microcapillary channel(s) 19 may be filled with fluid sample 31. In other embodiments the reactor module(s) 11 and sample transmission channel(s) 1 are arranged in such a way that fluid sample 31 does not fill the microcapillary channel(s) 19, when a fluid sample is filled into the inlets 5 and 6 etc. In this case the microcapillary channel(s) provides additional space for matter expansion or for the movement of matter.

As explained above, an expansion may result from changes in temperature, pressure or volume. A movement of matter may for instance occur as a result of matter expansion. Where for instance an isolation medium 33 is filled into the sample transmission channel(s) 1 after a fluid sample 31 has been filled therein, the reactor module 11 contains fluid sample 31, while the sample transmission channel 1 contains isolation medium 33. In this case the isolation medium may expand upon changing its aggregation state and cause a movement of the fluid sample in the reactor module. Additionally, the process of filling isolation medium 33 into the sample transmission channel(s) 1 may cause a slight movement of isolation medium into the inlet of the reaction chamber(s) of the reactor module(s) 11. The isolation medium thus displaces some fluid sample, causing it to move through the reactor module. As a consequence the microcapillary channel(s) 19 fill with the fluid sample. In such embodiments the microcapillary channel(s) 19 therefore assist the fluid isolation chamber(s) 23 in its/their function.

In some embodiments an outlet of the reaction chamber(s) is equipped with two microcapillary channels. In other embodiments reaction chamber(s) are equipped with two outlets, each outlet providing one microcapillary channel 19 that is in fluid communication with the same fluid isolation chamber 23 as the other microcapillary channel. These two microcapillary channels may again be located on distal portions of the reaction chamber 15 with respect to the inlet 12 (which may be a channel 13, for example). In some embodiments the two microcapillary channels may furthermore be arranged symmetrically providing a communication with two inlets 20 of a fluid isolation chamber, optionally over the same distance. Such an arrangement is exemplarily illustrated in FIG. 4A. In other embodiments the two microcapillary channels may provide a communication with inlets 20 of two separate fluid isolation chambers. Such an arrangement is exemplarily illustrated in FIG. 4B.

As indicated above, the shape of the reaction chamber(s) may be selected in such a way that the formation of air bubbles during the process of filling with fluid sample 31 is avoided or prevented. In embodiments where the reaction chambers are equipped with two outlets that provide microcapillary channels, further examples of means to avoid the formation of air bubbles include, but are not limited to, walls/sides adjacent to the respective outlets with a convex shape. Such shape may particularly be selected for the walls or wall portions 17 that extend between the two outlets providing the microcapillary channels 19 (see FIGS. 4A and 4B). A convex shape may for instance comprise hemispherical, semi-elliptical or polygonal protrusions.

As indicated above, microdevices such as the one of the present invention are often made of glass or polymeric substrates. Generally, the substrate of the microdevice of the present invention may be made of or comprise any material that is compatible with the desired analysis of a respective fluid sample. Depending on the desired method of analysis, the material may be required to be translucent or non-fluorescent. Examples of materials, which the substrate used for the microdevice of the present invention may comprise, thus include, but are not limited to, silicon, quartz, glass, plastic (such as thermoplastics), elastomer (such as PDMS or elastic silicone rubber), metal and composites thereof.

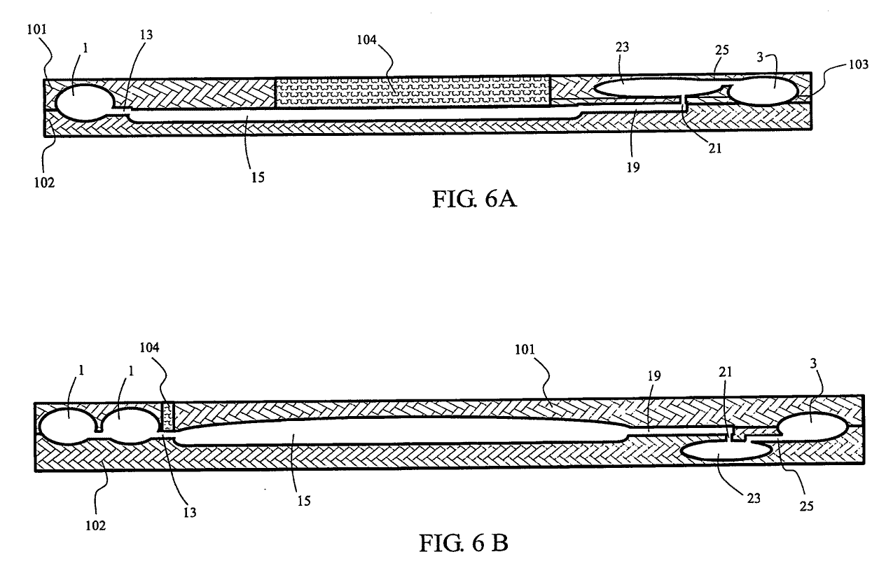

In some embodiments, some or all components of the device of the present invention may be generated by etching onto a substrate. In other embodiments, a number of components may be incorporated into the apparatus or substrate, including an optional covering layer (see below). In yet other embodiments, the device may be built up of several substrate layers (e.g. 101 to 104 in FIG. 6 or 100 to 103 in FIG. 7B) so as to allow an assembly during manufacture or before use. Such substrate layers may be of any shape, thus for instance forming substrate portions of various thickness, including portions that span the entire height of the device. The respective substrate layers may comprise the same or different substrate materials. Typically, the assembly of these substrate layers and/or portions will include a sealing, so as to allow for a complete and tight connection of the different parts. A respective sealing may for instance be performed by a glue. Any glue that is compatible with desired measurements of a fluid sample in the reactor module(s) may be used. In some embodiments the glue may thus need to be non-fluorescent or translucent. In other embodiments, for example where it is desired to analyse biological fluid samples containing living cells over a period of 24 hours or more, the glue may need to compatible with autoclavation.

Optionally one substrate layer of the device of the present invention forms a covering layer, which closes any part of the device. The covering layer may for instance cover a channel or a chamber, thus for example sealing a reaction chamber 15 (see e.g. substrate layer 104 in FIG. 6A) or a reaction chamber inlet channel 13 (see e.g. substrate layer 104 in FIG. 6B). It may also seal one or more of the loading ports 4 to 9. Accordingly, the covering layer is typically located on the top of the device. In such embodiments it may close the entire surface(s) of the substrate layer(s) below, or close all of the respective surface(s) with the exception of loading ports, such as loading ports 4 to 9. In other embodiments the covering layer may optionally provide venting holes, for instance in order to allow the escape of evaporated solvent. One or more compartments of the device, such as loading ports 4 to 9, venting holes or the reaction chambers 15, may alternatively be equipped with a separate sealing means, as for instance a lid. Such separate sealing means may be able to open and close and may be activated mechanically, electrically, and/or magnetically.

A covering layer and additional separate sealing means may thus generally serve the function of providing three dimensionally closed or controllably closable compartments. This function is completed in conjunction with the usage of the above mentioned additional sealing material that need not be part of the device. Using this combination, the whole or any part of the device may thus, if desired, be hermetically sealed, i.e. air tight. The covering layer may furthermore comprise any of the functional compartments of the device, such as for instance the sample transmission channel(s) 1 or the multi-functional channel(s) 3, or parts thereof. Hence, the covering layer may be build up in such a way as to complete the device, when placed onto the substrate.

The covering layer and additional separate sealing means may be of any suitable rigid or semi-rigid material. In some embodiments the same material as for the substrate may be used. In other embodiments a self-sealing material such as a rubber or an elastomer may be used, so as to allow for a penetration, for instance by mechanical, electrical, chemical or magnetic means. As an example, a penetration of a covering layer may be performed with the needle of a syringe. Where a self-sealing material is used, this will prevent the formation of for instance a remaining hole by self-closing.

The invention is further directed to a method of detecting an analyte in a fluid sample using the device of the present invention. The method of detecting an analyte typically comprises methods of self-distributing and/or transmitting, enclosing and/or isolating, and subsequently, analysing fluid samples using the device of the present invention. As used herein, the term ‘detecting’, detect’ or ‘detection’ refers broadly to any measurement which provide an indication of the presence or absence, both qualitatively and/or quantitatively, of an analyte. Accordingly, the term encompasses quantitative measurements of the concentration of an analyte in a fluid sample, as well as qualitative identification of the different types of analytes that are present in a given sample, or the behaviour of a particular analyte in a given environment is observed, for instance.

The invention is also directed to methods of distributing, enclosing or storing a fluid in an enclosed space using the device of the present invention. Fluid samples can be self-distributed and/or transmitted through micro-scale fluid channels within the device by establishing sufficiently large capillary forces to drive the bulk movement of the fluid sample, such that the fluid sample distributes itself within the device, without the need for auxiliary pumps or valves.

The present method of detecting an analyte in a fluid sample comprises the steps of providing a device having the features as defined in above-described device according to the invention, and then loading a fluid sample which is to be analysed into the device. Fluid sample can be loaded directly into any suitable part of the device, such as the fluid transmission channel or the reaction chamber. Said loading may also be carried out indirectly, for example by introducing fluid sample into the sample transmission channel via a loading port or receiving well which is fluidly connected to it. The loading of the fluid sample into the device is typically carried out using dispensing instruments such as an injection pipette or a dropper that can manually or robotically dispense small quantities of fluid into a receiving chamber in the device, such as loading ports 5, 6, or 9 (see above). The fluid sample may be introduced at one or several such receiving chambers present in the device. In some embodiments, capillary pressure generated from reduced surface tension at the solid-liquid interface between the fluid sample 31 and the walls of the channel facilitates the flow of fluid sample through the sample transmission channel 1.

In one embodiment, surface affinity between the fluid sample and the walls of various fluid channels within the device is varied to control fluid flow within the device, thereby providing a means to control the flow behaviour of a fluid sample within the device, without requiring the use of valves or any other fluid control devices. In other words, by combining the use of different capillary forces and surface affinities, a variety of distributions profiles can be established. Such control is desirable for establishing efficient loading procedures. For example, loading procedures which minimise spillage or which minimise contamination of the fluid sample during the loading process can be developed based on said fluid control. For example, if it is desired to prevent an aqueous fluid sample flowing in a first channel from entering a second channel, the walls of the second channels can be rendered hydrophobic (e.g. by coating with a hydrophobic layer) so as to reduce the ease with which the aqueous fluid sample flows into the second channel. Alternatively, if it is desired to induce the aqueous fluid sample to enter into the second channel, the second channel may be rendered more hydrophilic than the first channel in order increase the ease with which the fluid sample enters the second channel. The former method can be used, for example, to achieve partial fluid sample distribution within the reactor module (i.e. fluid sample is stopped from entering certain channels within the reactor module) while the latter method can be used to achieve complete distribution of fluid within the reactor module.

In a presently preferred embodiment, the device for detecting an analyte comprises a plurality of reactor modules in which the loading step is carried out to effect a partial fluid sample distribution profile within the reactor module. In order to achieve said partial distribution of within each reactor module, the at least one outlet of the reaction chamber comprises at least one microcapillary channel which is rendered relatively less hydrophilic than the reaction chamber or even hydrophobic, thereby preventing fluid sample that is of a hydrophilic nature from entering into the at least one microcapillary channel.

Subsequently, if it is desired to effect a complete distribution of a fluid sample within each reactor module, sealing material can be introduced into the inlet 12 (also known as the “inlet port” or “receiving well”) of the reaction chamber 15. In one embodiment, the inlet (or neck in some embodiments) of the reactor module is rendered receptive to the sealing material so that the sealing material enters the neck and displaces some fluid sample into the microcapillary channel. In this manner, the complete distribution of a fluid sample is carried out as a two-step procedure in which a fluid sample is first partially distributed within the reactor module by the loading step, and then completely distributed only when the step of sealing the sample transmission channel material is carried out.

If a one-step distribution procedure is desired, the complete distribution of fluid sample within the reactor module is preferably achieved within the loading step. In order to achieve said complete distribution in a one-step procedure, the at least one outlet of the reaction chamber comprises at least one microcapillary channel which is rendered similarly hydrophilic or more hydrophilic than the reaction chamber, thereby allowing fluid sample that is of a hydrophilic nature to enter into the at least one microcapillary channel. In this case, there is no need for the sealing material to be used for pushing fluid sample into the microcapillary channel.

Alteration of surface characteristics of the walls of any part device of the present invention e.g. the microcapillary channel or the neck of the reaction chamber, is typically achieved by chemical means. For example, any suitable reagent that is capable of lowering surface tension at the solid-liquid interface may be pre-loaded into the sample transmission channel or pre-coated onto the walls of the channel in order to promote the flow of fluid sample 31 through the channel. In general, such reagents serve to increase attractive forces between the fluid sample 31 and the walls of the channel. Examples of suitable reagents include, but are not limited to, cationic, anionic, nonionic, and zwitterionic surfactants such as sodium dodecyl sulfate (SDS), cetyltrimethyl bromide (CTAB), Triton-X100 and 3-[(3-Cholamidopropyl) dimethylammonio]-1-propanesulfonate (CHAPS), provided that the reagent does not interfere with the analyte detection reaction carried out later on, or with the collection of the reaction data.

As the fluid sample flows along the fluid transmission channel, it enters the inlet of the reaction chamber and fills the reaction chamber 15. Thereafter, a sealing material is introduced into the sample transmission channel(s) 1 and the multi-functional channel(s) 3 in order to isolate the fluid sample within the reaction chamber and to minimize contact between the fluid sample 31 and the atmosphere. The step of introducing the sealing material may be carried out in any sequence, either first introducing the sealing material into the sample transmission channel(s) 1 and then the multi-functional channel(s) 3, vice versa, or it can also be carried out simultaneously.

Any suitable sealing material may be used for sealing the sample transmission channel and the multi-functional channels, including high density liquids or gel-like substances derived from polymers, as well as gases such as water vapour which can be introduced to minimise evaporation of water from the fluid sample, as well as inert gases such as nitrogen and argon. In general, the selection of the sealing material may depend on the nature of the fluid sample. For example, the sealing material may be any substance that is in a different physical state from the fluid sample, or it can be any substance that is substantially not miscible with the fluid sample 31. For example, if the fluid sample to be tested is an aqueous liquid, the suitable sealing material is preferably a hydrophobic substance. Contemplated materials include but are not limited to wax, oil, plastics, silicones, and phase change polymers which can solidify over a range of temperatures, preferably but not limited to temperatures slightly above room temperature to temperatures of around room temperature. Alternatively, if a hydrophobic substance is tested, hydrophilic substances may be used as sealing materials. In other embodiments, the sealing material is derived from a polymer precursor which may optionally be treated by any suitable means, such as UV irradiation, heating, cooling or exposure to air, in order to turn the precursor into the sealing material. In yet other embodiments, the sealing material comprises an adhesive which solidifies after the evaporation of the solvent in which the adhesive is prepared, for instance. In this embodiment, venting holes may be provided to allow the escape of evaporated solvent.

In the embodiment where the sealing material is a polymer which is derived from a polymer precursor, the step of sealing the fluid transmission channel(s) and the multi-functional channel(s) comprises, firstly, introducing a polymer pre-cursor into the sample transmission channel(s) and multifunctional channel(s), and secondly, polymerising the polymer pre-cursor to form a polymer that can be used for sealing the reactor module. Polymer precursors are preferably present in the liquid phase at room temperature and can be treated or reacted to form solid or gel-like polymers. Furthermore, polymer precursors have suitable physical characteristics (e.g. weak intermolecular forces, low viscosity and low surface tension) that allow it to be flow within milli-scale or micro-scale fluidic channels. As used herein, the term ‘polymer precursor’ include monomers that can be polymerised to form solid phase or gel-phase polymers, as well as liquid or gel-phase polymers that can be solidified by converting the polymer into the solid phase or gel phase by curing. Exemplary polymer-precursors include phase change plastics, thermally curable polymer (thermoplastic) liquids e.g. linear, cyclic or aromatic hydrocarbons, cyanoacrylates or siloxanes such as polydimethylsiloxane (PDMS), silicone elastomers, and liquid silicone precursors; ultraviolet light (UV) curable polymers such as polyvinylchloride, polyacrylate, and polyurethanes, etc.

Sealing material can be introduced into the device via any of the following non-exhaustive list of methods: positive pressurization, electro-osmosis, suction, capillary flow and electrowetting. The means may be used for carrying these methods include microfluidic injectors, electrowetting on dielectric film, piezoelectric micropumps, etc.

After sealing material has been deposited in the channels, at least one analyte detection reaction is carried out in order to provide at least one qualitative or quantitative data relating to the analyte. The data obtained may be used for a variety of purposes, for instance, to infer the presence or absence of an analyte, or to detect the concentration of a particular analyte present in the fluid sample.

Generally, the selection of reaction(s) to be carried out in order to detect a respective analyte depends on the type of analyte to be detected, taking into account the characteristics of the analyte which allows for its detection. The reactions that may be carried out in the present method can be classified generally either as core processes or subsidiary processes. Core processes refer to reactions which involve an analyte in the fluid sample and which yields the desired qualitative or quantitative information (data) about the analyte. Such data may directly or indirectly indicate the detection of a targeted analyte. Subsidiary processes include the mixing of fluid samples with analytical reagents, homogenizing procedures to render heterogeneous samples suitable for analysis, and the removal of interferents via separation procedures such as washing, for example.

Core processes include, for instance, binding reactions between the analyte that is targeted for detection and an indicator compound which provides a detectable signal to indicate positive detection of the analyte. Examples include for instance immunochemical reactions such as an Enzyme-Linked Immunosorbent Assay EUSA), which is well known to the person skilled in the art. Other examples include enzymatic reactions, which rely on the generation or consumption of molecules with a characteristic absorbance. Such reactions are well known to the person skilled in the art and involve for instance a redox change of molecules such as Nicotinamide Adenine Dinucleotide (NAD/NADH). Yet another example is the binding reaction between a targeted DNA sequence and its complementary DNA or a fragment thereof, labelled with a fluorophore, whereby a fluorescent signal is produced if the test sample contains the target DNA sequence.

In one embodiment in which the detection of nucleic acids is to be carried out, the core process of nucleic acid amplification reaction is performed in one of the reactor modules 11. The reactor module may be subject to a thermal condition required for DNA amplification. Such thermal conditions include thermal cycling required for polymerase chain reaction.

In one embodiment, the method of the invention provides at least one qualitative or quantitative data which provides at least one of a colorimetric, fluorometric or luminescent result relating to the analyte present in the fluid sample. If a calorimetric result is desired, for example for the detection of a protein analyte, suitable dyes may be used to stain any protein present in the fluid sample. An example of a usable dye can be obtained from sulfo-rhodamine B (SRB) dissolved in acetic acid. Subsidiary processes such as washing may be required to remove unbound dye may be removed by washing, and other subsidiary process may be required to extract protein-bound dye for determination of optical density in a computer-interfaced microtiter plate reader. Where a fluorometric result is desired, fluorescent dyes may be used. For instance, such dyes can be used in conjunction with tracing techniques to provide a means of measuring the rate of fluid flow through fluid channels in the device. The fluorometric result can also be derived from fluorescence provided by either the binding of a fluorophore directly to a targeted analyte, or the binding of a fluorophore-labelled compound to the targeted analyte. In a further embodiment, probes that are bound with at least one fluorophore, enzyme, or component of a binding complex is used for the detection of the analyte.

The device of the invention that is employed in conjunction with the present inventive method may be designed with any number of reactor modules and sample transmission channels, and multifunctional channels as required, depending on the reactions to be carried out for detecting the analyte. In one embodiment, where a multitude of several of core and subsidiary processes are to be carried out, a device having a plurality of interconnected reactor modules can be used. The plurality of reactor modules may be arranged into any suitable configuration to facilitate fluid sample distribution. For example, the reactor modules may be arranged into rows of which are connected to a common sample transmission channel and a common multi-functional channel. One row of reactor modules may furthermore communicate with other rows of reactor modules via fluid interconnections between the multi-functional channels and fluid transmission channels of separate rows of reactor modules. On the other hand, if a simple core process is to be carried out, a device having only a single reactor module can be used. Where a plurality of reactor modules 11 are present, the step of loading the fluid sample into the device of the invention can be carried out such that the reactor modules are filled simultaneously, meaning that the fluid sample is introduced into each reactor module at approximately the same time. On the other hand, it is also possible to have the reactor modules filled in sequence, meaning that one reactor module after another is filled.

The present method can be carried out to detect analytes from biological or non-biological material. Examples of non-biological material include, but are not limited to, synthetic organic or inorganic compounds, organic chemical compositions, inorganic chemical compositions, combinatory chemistry products, drug candidate molecules, drug molecules, drug metabolites, and any combinations thereof. Examples of biological material include, but are not limited to, nucleotides, polynucleotides, nucleic acids, amino acids, peptides, polypeptides, proteins, biochemical compositions, lipids, carbohydrates, cells, microorganisms and any combinations thereof.

Examples of nucleic acids are DNA or amplified products from the processing of nucleic acids for genetic fingerprinting, e.g. PCR. Examples of microorganisms include for instance pathogens such as bacteria or virus, or cancerous cells. Such analytes can originate from a large variety of sources. Fluid samples that may be analysed using the present method include biological samples derived from plant material and animal tissue (e.g. insects, fish, birds, cats, livestock, domesticated animals and human beings), as well as blood, urine, sperm, stool samples obtained from such animals. Biological tissue of not only living animals, but also of animal carcasses or human cadavers can be analysed, for example, to carry out post mortem tissue biopsy or for identification purposes, for instance. In other embodiments, fluid samples may be water that is obtained from non-living sources such as from the sea, lakes, reservoirs, or industrial water to determine the presence of a targeted bacteria, pollutant, element or compound. Further embodiments include, but are not limited to, dissolved liquids, suspensions of solids (such as microfluids) and ionic liquids. In yet another embodiment, quantitative data relating to the analyte is used to determine a property of the fluid sample, including analyte concentration in the fluid sample, reaction kinetic constants, analyte purity and analyte heterogeneity.

Any bacteria, virus, or DNA sequence can be detected using the present invention for identifying a disease state. Diseases which can be detected include communicable diseases such as Severe Acute Respiratory Syndrome (SARS), Hepatitis A, B and C, HIV/AIDS, malaria, polio and tuberculosis; congenital conditions that can be detected pre-natally (e.g. via the detection of chromosomal abnormalities) such as sickle cell anaemia, heart malformations such as atrial septal defect, supravalvular aortic stenosis, cardiomyopathy, Down's syndrome, clubfoot, polydactyl), syndactyl), atrophic fingers, lobster claw hands and feet, etc. The present method is also suitable for the detection and screening for cancer.

Apart from the detection of nucleic acid based analytes, the present invention may also be employed for the detection of pharmaceutical compounds such as drugs. This aspect of the invention can be used for drug screening or for determining the presence of a drug in a urine or blood sample.

Other objects, advantages and features of the present invention will be apparent from the following detailed description of some embodiments of the invention with reference to the attached drawings and examples, in which:

FIG. 1 is a plan view of a device according to the invention in which a sample transmission channel 1 and a multi-functional channel 3 are connected to a reactor module 11 comprising a reaction chamber 15 and a fluid isolation chamber 23.

FIG. 2 is a plan view of two embodiments of the device in which the reaction chamber 15 is in fluid communication with a fluid isolation chamber 23 via a port. While in the embodiment depicted in FIG. 2A inlet 12 and outlet 18 of the reaction chamber 15 are located on proximal and distal portions of the reaction chamber 15, FIG. 2B depicts an embodiment with a perpendicular arrangement of the respective inlet 12 and outlet 18. It should be noted that sample transmission channel 1 and multi-functional channel 3 do not need to be horizontally on the same level within the device. A respective difference is not visible in a plan view.

FIG. 3 depicts plan views of four other embodiments of the device in which the outlet of the reactor module comprises a microcapirary channel 19, which is in fluid communication with the fluid isolation chamber 23 via a port 21. The microcapillary channel 19 contains an opening 22, which leads into the port 21.

In FIG. 3A the inlet 12 of the reaction chamber 15 has the form of an opening, while in FIG. 3B it has the form of a channel 13. Furthermore inlet 12 and a microcapillary channel 19 are located sidelong relative to each other in FIG. 3A, while they are located on opposing walls in FIG. 3B.

FIG. 3D shows an embodiment, in which channel 13 is branched, and where it provides bevelled portions 10. FIG. 3C depicts an embodiment, where the reaction chamber inlet provides a chamber 14. Furthermore, in the embodiments shown in FIGS. 3C and 3D two reaction chambers 15 as well as two fluid isolation chambers 23 are present within one reactor module. Reaction chambers 15 and fluid isolation chambers 23 are arranged in parallel, horizontally adjacent two the second respective compartments. It should be noted that this embodiment may also be defined as comprising two parallel reactor modules, which share a common inlet in form of inlet chamber 14.

Furthermore, the embodiment depicted in FIG. 3D comprises two multi-functional channels 3, which are not in direct connection with each other.

FIG. 4 is a plan view of two further embodiments of the device in which the inlet of the reactor module comprises a neck. Two microcapillary channels 19 connect the reaction chamber 15 to at least one fluid isolation chamber 23, which is in turn connected to a multifunctional channel 3 via an outlet channel 25. While FIG. 4A shows an embodiment with one fluid isolation chamber 23, FIG. 4B shows an embodiment with two fluid isolation chambers. In the embodiment shown in FIG. 4B each microcapillary channel 19 is connected to a different fluid isolation chamber 23.

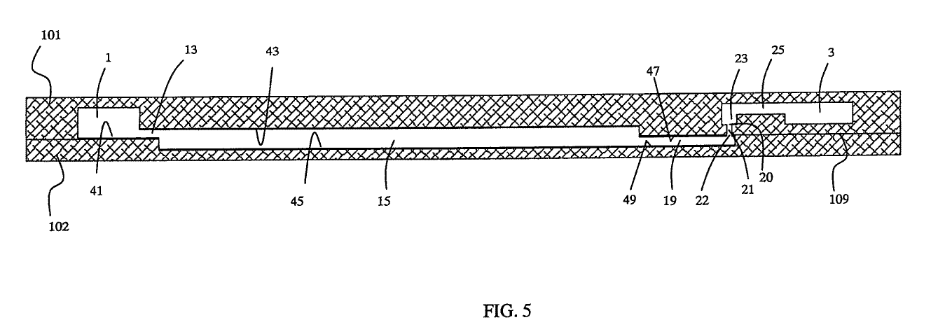

FIG. 5 shows a side view of another exemplary device in which at least one microcapillary channel 19 is present. In the depicted embodiment, the fluid isolation chamber 23 is situated directly above the microcapillary channel 19 and is connected to it via a perpendicular port. In the depicted embodiment, the device furthermore comprises two substrate layers 101 and 102.

FIG. 6 shows side views of two other exemplary devices in which at least one microcapillary channel 19 is present. In these embodiments the device furthermore comprises several substrate portions, 101 to 104 in FIG. 6A and 101, 102 and 104 in FIG. 6B, all of which are forming a layer. Layers 101 and 102 horizontally stretch across the device entirely. Layers 101 and/or 104 may be a covering layer. In case of layer 104 forming such a covering layer, it forms a layer on top of reaction chamber 15, covering a part of it. Such a covering layer may be of a self-sealing material.

While in the embodiment depicted in FIG. 6A the fluid isolation chamber is located on a vertically higher level than the reaction chamber, it is located on a vertically lower level in the embodiment shown in FIG. 6B. The arrangement of the compartments of the reactor module and the multi-functional channel 3 nevertheless prevents the flow of fluid from the reaction chamber 15 into the multi-functional channel 3 and vice versa. Furthermore, FIG. 6B shows an embodiment of a device comprising two sample transmission channels 1.

FIG. 7 shows a side view of devices of the invention in which two (FIG. 7A) or three (FIG. 7B) reaction chambers 15 are present and are arranged one above the other within one reactor module. In the embodiment depicted in FIG. 7A the reaction chambers are connected to a common fluid isolation chamber 23 via ports 21. The embodiment depicted in FIG. 7B contains two fluid isolation chambers 23, two outlet channels 25 and two multifunctional channels 3. While the reaction chambers 15 are located exactly on top of each other in the embodiment shown in FIG. 7A, in the embodiment depicted in FIG. 7B they are located at horizontally different, although overlapping, positions. It should be noted that the inlet channels 13 of the reaction chambers as well as the microcapillary channels 19 need not be located exactly on top of each other. Such embodiments were selected for illustrative purposes only, as a cross section would otherwise not depict all of the respective channels.

FIG. 8A shows a cross-sectional view of the exemplary device of FIG. 5 at the location of the 2 microcapillary channels 19. FIGS. 8B, 8C, 8D, 8E and 8F show different permutations of a surface treatment, e.g. a coating that may be applied to the walls of a microcapillary channel 19. FIGS. 8G, 8H, 8I, 8K and 8L depict other embodiments of respective microcapillary channels 19 with a surface treatment such as a coating applied to an inner surface. It should be noted that a cross-sectional view of other channels of the device, such as the multi-functional channel or the sample transmission channel may resemble the depicted embodiments.

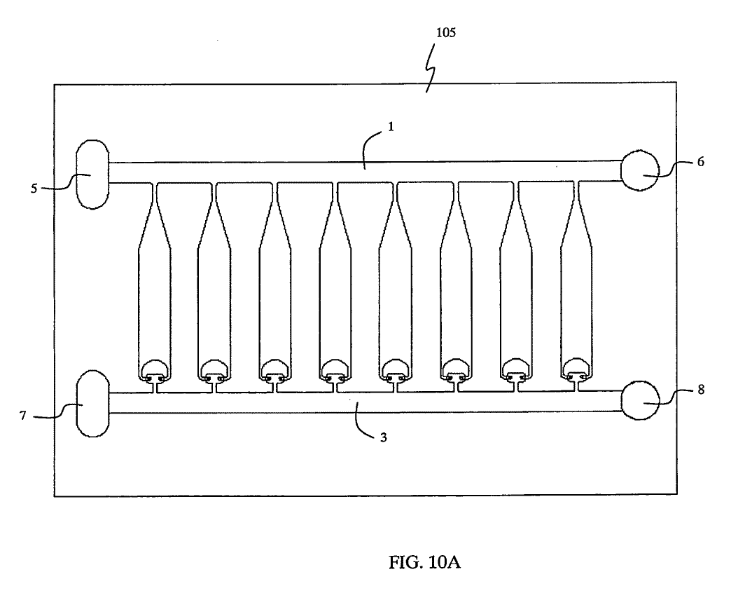

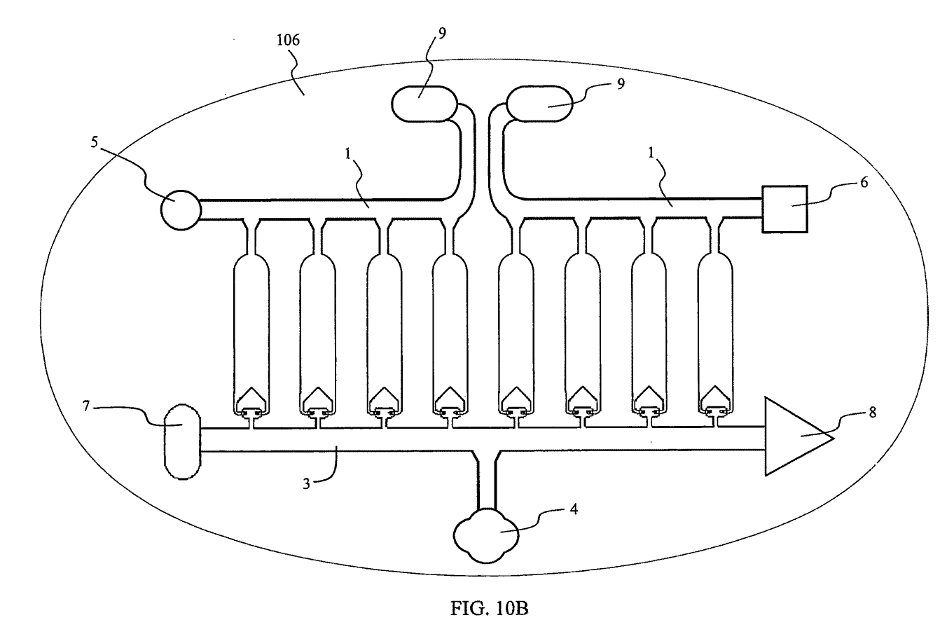

FIG. 9 shows a cross-sectional view of the exemplary device of FIG. 5 at the location where the two microcapillary channels 19 are connected to the fluid isolation chamber 23, each via a port 21. The depicted cross-section is thus taken from the perspective of a viewer looking through the reaction chamber or from the outlet of the fluid isolation chamber.

FIG. 10 shows a plan view of two embodiments of the device in which a plurality of reactor modules are connected to a common fluid sample transmission channel 1, as well as a common multifunctional channel 3. FIG. 10A depicts an embodiment, where the plurality of reactor modules is located on substrate layer(s) 105, which are of rectangular shape when seen in a plan view. In the embodiment depicted in FIG. 10B, the respective substrate layer(s) 106 are of ovoid shape in this perspective.

In FIG. 10A both the sample transmission channel 1 and the multi-functional channel 3 are linear and straight. In FIG. 10B, two sample transmission channels 1 are present, which are bent, and the multi-functional channel 3 is branched. In the embodiment shown in FIG. 10B, the multi-functional channel 3 is thus in fluid communication with the three loading ports 4, 5, and 6. In the embodiment shown in FIG. 10B, the plurality of reactor modules is furthermore in fluid communication with the same multi-functional channel 3, while the right half of the reactor modules is in fluid communication with the right sample transmission channel, and the left half of the reactor modules is in fluid communication with the left sample transmission channel.

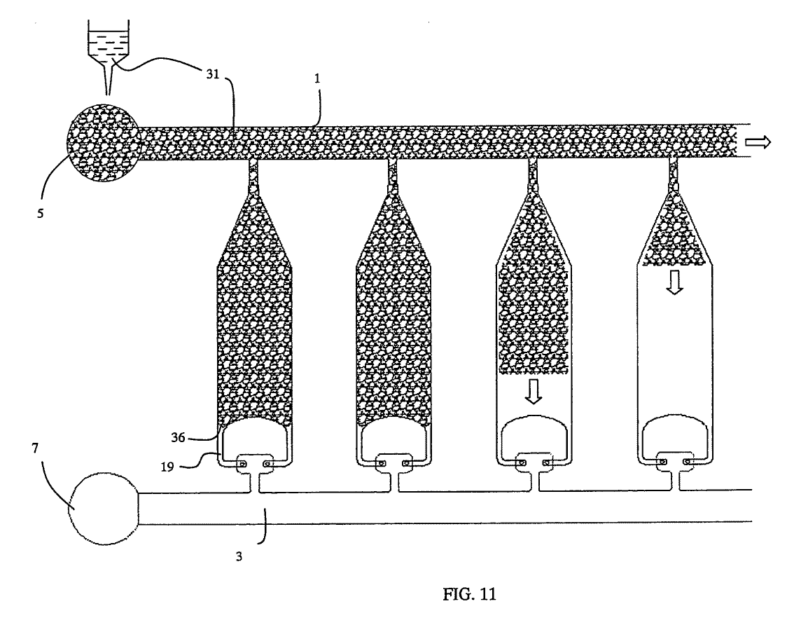

FIG. 11 depicts the loading of fluid sample into one embodiment of the device of the invention having four reactor modules. The left two reaction chambers are already filled with fluid sample 31, while the two reaction chambers on the right are currently in the process of being filled. It should be noted that in some embodiments of the device of the present invention the microcapillary channels 19 are not filled with fluid at this stage.

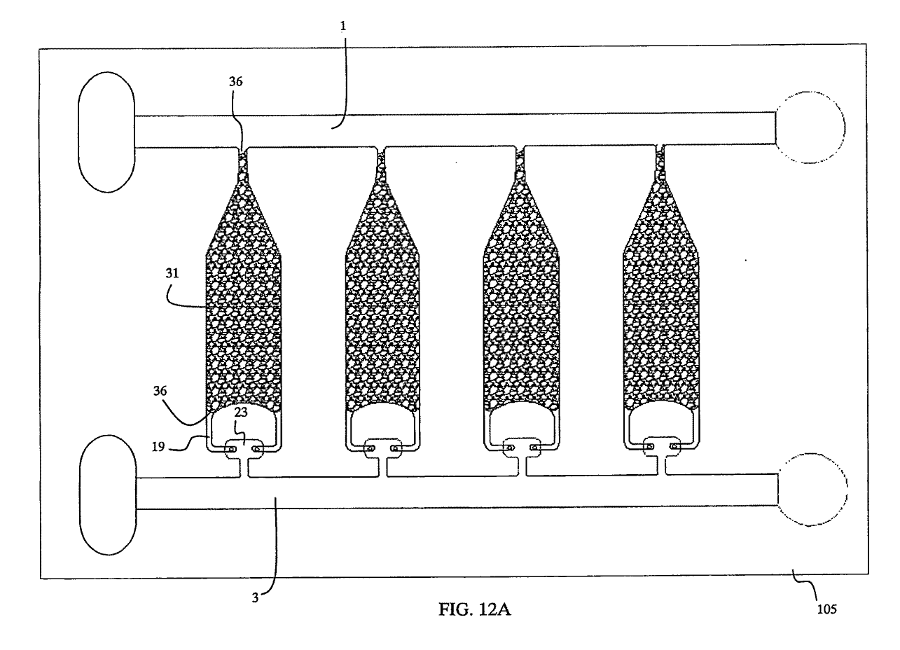

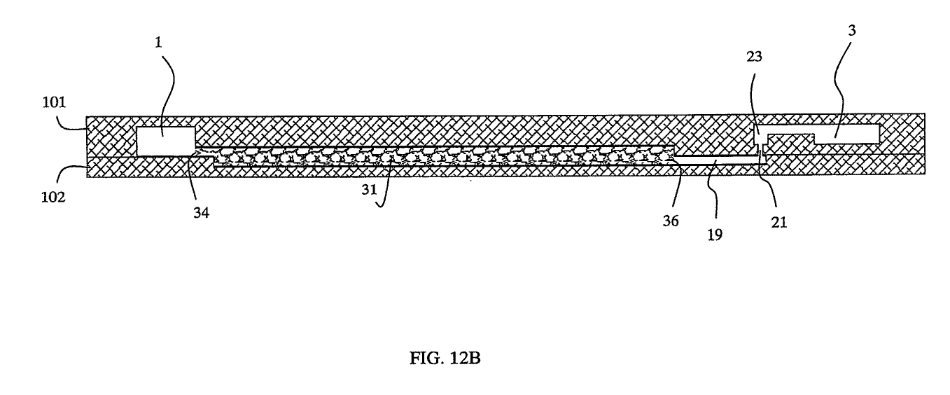

FIG. 12A depicts the completed distribution of fluid sample 31 into the four reactor modules. The distribution profile of the fluid sample of the depicted embodiment is such that no fluid sample enters the microcapillary channel even after loading is complete.

FIG. 12B shows a side view of the exemplary device of FIG. 12A in which the distribution of fluid sample is completed.

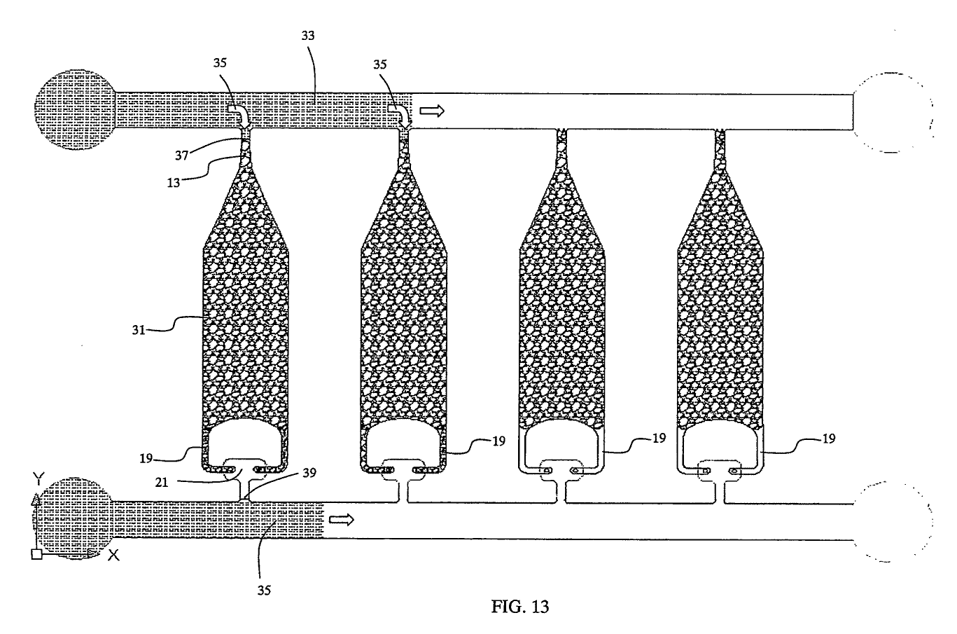

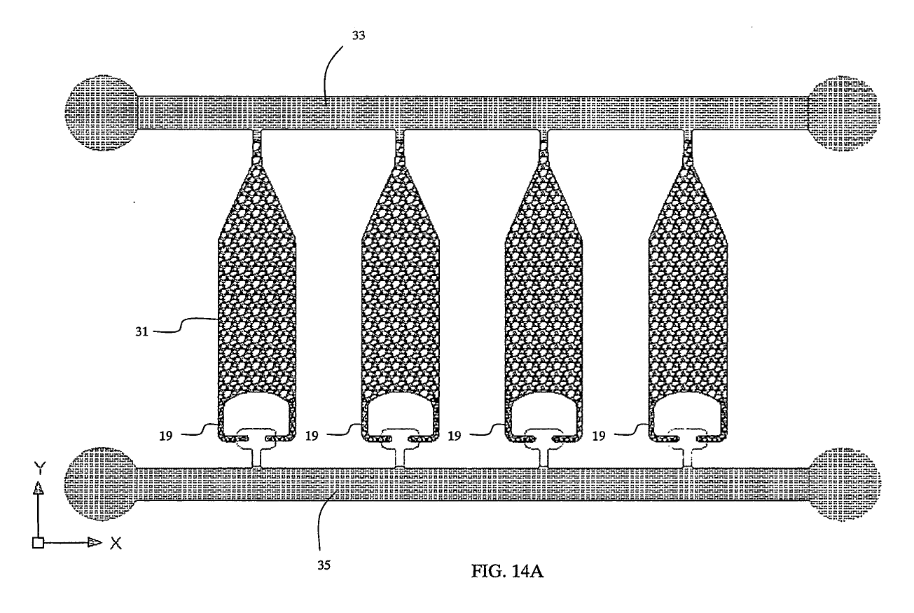

FIG. 13 depicts the sealing of the sample transmission channel and the multifunctional channel with sealing material 33. In embodiments where the microcapillary channels 19 are not yet filled with fluid, the capillary forces may cause the entry of sealing material 33 into the inlet channel 13 of the reaction chambers. Such flow in turn causes a filling of the microcapillary channels 19 with fluid sample 31. The arrangement of microcapillary channel(s) 19, port(s) 21 and the fluid isolation chamber however prevents the entry of fluid sample into the fluid isolation chamber. Accordingly, the sealing material 33 is prevented from further flowing into the inlet channel 13 of the reaction chamber 31.

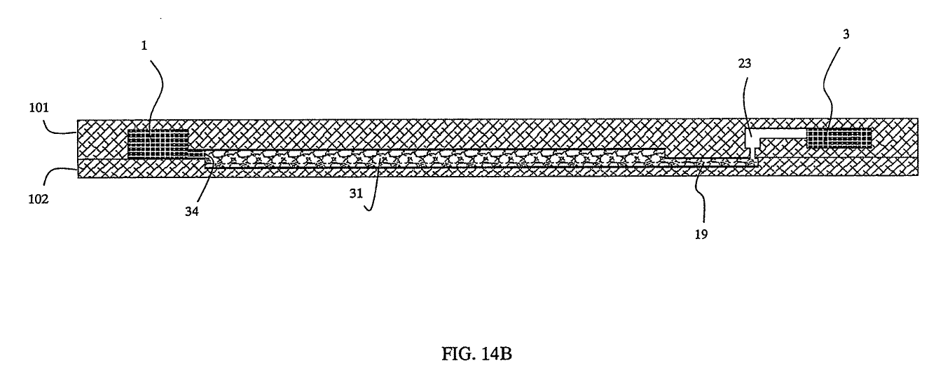

FIG. 14A depicts completed distribution of sealing material into the sample transmission channel and the multifunctional channel. In this embodiment a small amount of sealing material has entered the reactor module from the sample transmission channel and displaces some of the fluid sample into the microcapillary channel. However, no fluid sample has entered the reaction chamber. FIG. 14B shows a side view of the exemplary device of FIG. 14A in which the distribution of sealing material is completed.

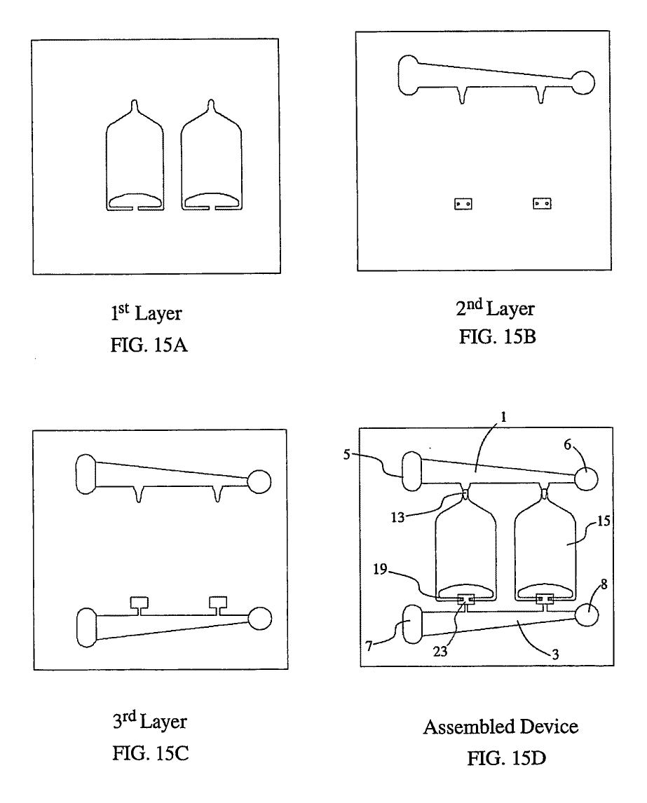

FIGS. 15A, 15B, and 15C show the schematic of three substrate layers that can be assembled to form one embodiment of the device according to the invention as shown in FIG. 15D.

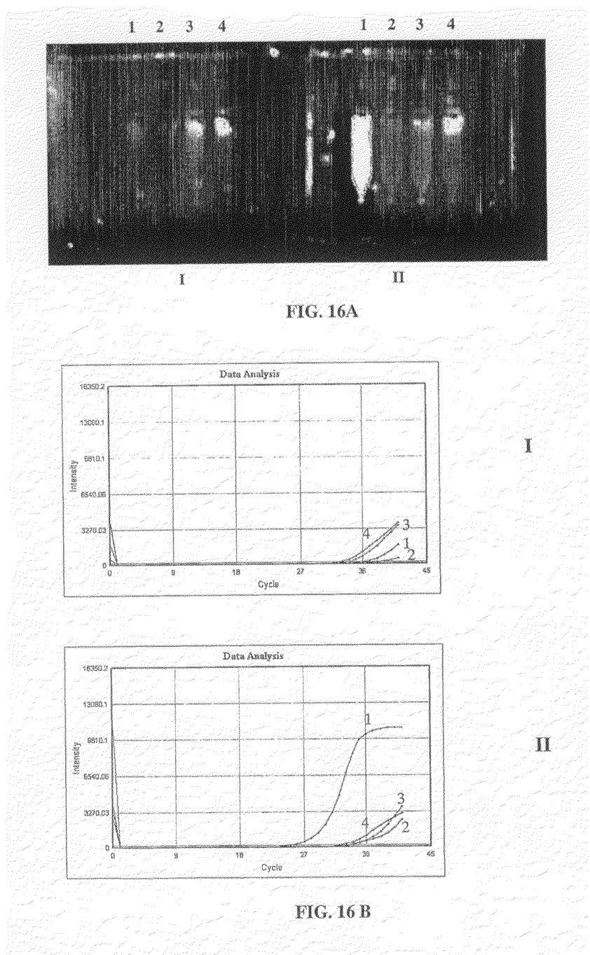

FIG. 16A depicts a photograph of the fluorescence emission images of a sample analysed with a device of the present invention.

FIG. 16B depicts an exemplary use of a device of the present invention in the real-time fluorescent acquisition profiles of the reaction chambers during the course of the reaction.

### EXEMPLARY EMBODIMENTS OF THE INVENTION

FIGS. 1, 2 and 3 show exemplary fluid microstructures of a device according to the invention. In these examples, a sample transmission channel 1 is connected to a reactor module 11 via inlet 12. The reactor module comprises a reaction chamber 15 and a fluid isolation chamber 23 connected to the outlet of the reaction chamber 15. In FIG. 1, the fluid isolation chamber 23 is directly connected to the outlet of the reaction chamber 15. FIG. 2 shows an alternative configuration in which the reaction chamber 23 is connected to the reaction chamber 15 via a port 21. In the embodiment shown in FIG. 2A, both the inlet and the outlet of the reaction chamber 15 are located along its longitudinal axis. FIG. 2B depicts an embodiment, where inlet and the outlet of the reaction chamber 15 are located sidelong toward each other. FIG. 3 shows yet another embodiment in which the reaction chamber is connected to the fluid isolation chamber via a single microcapillary channel 19 and a port 21. In the embodiment depicted in FIG. 3B, both the inlet and the outlet of the reaction chamber 15 are again located along its longitudinal axis, while inlet and the outlet are located sidelong toward each other in the embodiment depicted in FIG. 3A. The outlet of reaction chamber 15 is connected to the microcapillary channel 19 at a position to the right (FIG. 3A) or the left (FIG. 3B) of its longitudinal axis. It is also possible to connect the microcapillary channels at the anterior, posterior or at the middle of the reaction chamber 15. In all four examples, the fluid isolation chamber is connected to the multi-functional channel via an outlet in the form of an aperture 24 that is fluidly connected to the multi-functional channel 3.

FIG. 4 and FIG. 5 show preferred embodiments of the fluid microstructure in which the reaction chamber 15 is connected to at least one fluid isolation chamber 23 via two microcapillary channels 19, located at an end of the reaction chamber opposite to the location of the inlet 13, said inlet connecting the sample transmission channel 1 to the reaction chamber 15. A portion of the wall of the reaction chamber adjacent to the microcapillary channels 19 assume a convex configuration, as exemplified by convex-shaped wall 17. The term ‘convex-shaped wall’ as used herein refers to walls of the reaction chamber which protrude into the reaction chamber 15. Each of the two microcapillary channels comprises a bend 190 which links a first arm 191 to a second arm 192. Each second arm is connected to a substantially vertical port 21 which is in turn connected to fluid isolation chamber 23 situated above the microcapillary channels (cf. FIG. 5). The fluid isolation chamber 23 is connected via an outlet 25 to the multi-functional channel 3. In this embodiment, the outlet 25 is in the form of a channel

A convex-shaped wall 17 reduces the tendency of air-bubbles forming in the fluid sample when the fluid sample is introduced into the reaction chamber. In general, the tendency of air-bubbles forming in the fluid sample is reduced when the walls of the reaction chamber have smooth or rounded edges. Air bubbles typically form within the reactor module due to the presence of regions in the reaction chamber (e.g. sharp edges on the walls of the reaction chamber) which induce fluid turbulence. Although a convex wall is preferred, it does not preclude the possibility of using other alternative configurations, such as a level wall as well as irregularly shaped walls which may nevertheless work.

The side view of this embodiment can be seen in FIG. 5. A top substrate layer 101 is stacked on a bottom substrate layer 102. The top and bottom substrate layer meet at the interface 109. The surface of each substrate layer is etched with parts of microfluidic structures required in the device of the invention. When the top substrate layer is stacked in alignment onto the bottom layer, the etched microfluidic structures on each substrate layer fit together complementarily to form the microfluidic structure as shown in FIG. 5. In an alternative embodiment as shown in FIG. 6, two reactor modules are present, one module being in the top substrate layer and another module being in the bottom substrate layer.

The walls of the sample transmission channel may have lower affinity for the fluid sample than the walls of the reaction chamber in order to enhance the flow of fluid sample from the sample transmission channel into the by capillary force. Different surface affinities between the fluid sample and the channel walls in the device can be achieved by selecting suitable materials for fabricating the device. A typical hydrophilic material is glass, while hydrophobic materials are typically constructed from plastics. The surface characteristics (e.g. wetting characteristics) of these materials may be altered by various means such as mechanical, thermal, electrical or chemical treatment. A method commonly used in the art is a treatment with certain chemicals. For example, the surface of plastic materials can be rendered hydrophilic via treatment with dilute hydrochloric acid or dilute nitric acid. Alternatively, the surface properties of any hydrophobic surface can be rendered more hydrophilic by coating with a hydrophilic polymer or by treatment with surfactants. In cases where both top and bottom substrate layers comprise glass or any other hydrophilic material and the fluid sample is an aqueous fluid, the ceiling, floor or the side walls of the various channels and chambers may be rendered less hydrophilic than the reaction chamber by any method known in the art, including but not limited to plasma treatment or coating with a hydrophilic material. For example, a part or all of the surfaces of the transmission channel 1, the reaction chamber 15, and the microcapillary channel 19 may be coated with suitable reagents to render them more hydrophilic or less hydrophilic. Differences in affinity can be harnessed to control fluid flow and thus fluid distribution within the device. Taking the square-shaped, triangle shaped and circle shaped microcapillary channels as an example as shown in FIGS. 8A to 8L and FIG. 9, it can be seen that different portions of the walls 49 of the microcapillary channels 19 can be applied with hydrophilic and/or hydrophobic coatings (as identified by black shading and dashed shading) in order to provide different levels of affinities between the microcapillary channel and the fluid sample. Such coating may be applied before or after the assembly of the device (cf. FIG. 15).

FIG. 11 depicts one embodiment of a possible method of filling a sample transmission channel 1 and the reactor modules 11 with fluid sample 31 present in an exemplary device resembling the device shown in FIG. 10A, for example. Fluid sample 31 is dispensed into loading port 5 using a dropper or pipette or with any appropriate instrument for dispensing small amounts of liquid. As fluid sample 31 travels across the sample transmission channel 1, it enters the plurality of reaction chambers connected to the sample transmission channel, with the reaction chamber nearest to loading port 5 being filled first followed by the next reaction chamber and so on. In this manner, the reaction chambers are filled sequentially. The reaction chamber is filled to the brim of the inlet channel 13. Thereby subsequently a meniscus 34 is formed in case that the exact amount of a fluid sample, which matches the capacity of all reaction chambers of the device, has been allowed to enter loading port 5 (cf. FIG. 12 A). Alternatively, where an amount of fluid sample has been used that exceeds the capacity of the reaction chambers of the device, such excess fluid sample is drained into the loading ports 6 and 8. FIG. 12A shows the reactor modules in its completely filled state. In this embodiment, the fluid sample does not enter the microcapillary channels 19, and instead forms a meniscus 36 at the inlet of the microcapillary channel 19 (cf. FIG. 11). This distribution profile results from the walls of the microcapillary channels 19 having been modified to be less hydrophilic than the reaction chamber 15.

Sealing material 33, 35 are introduced into the sample transmission channel 1 and the multifunctional channel 3 to seal the fluid sample within the reactor module, as depicted in FIGS. 13 and 14. Some sealing material 33, 35 displaces fluid sample present in the inlet channel (see arrows 35), thereby forming a depressed meniscus 37 (interface between the sealing material and the fluid sample). The hydrostatic pressure from the sealing material is sufficiently large to overcome any affinity forces present, thereby displacing fluid sample into the microcapillary channels 19. As can be seen from FIGS. 14A and 14B, the fluid sample is present in the microcapillary channel 19 after the sealing material is introduced. The diameter of the port 21 linking the microcapillary channel 19 to fluid isolation chamber 23 is sufficiently small so that capillary flow in microcapillary channels 19 is disrupted. This disruption provides sufficient barrier to prevent the fluid sample from entering the port 21 and into the fluid isolation chamber 23 despite the hydrostatic pressure resulting from capillary flow in the microcapillary channels 19.

FIG. 15 depicts a master for fabricating a device of the present invention. FIGS. 15A to 15C show three substrate layers which are coated with Cr and Au, and thereafter with a photoresist. The patterns for the compartments of the device are created by photolithography. Subsequently, the respective compartments are created by etching. The respective assembled device shown in FIG. 15D broadly resembles the embodiment depicted in FIGS. 10A and 12. Alternative processes may be employed such as methods that employ microfluidic photomasks (Chen, C et al., Proc. Natl. Acad. Sci. USA. (2003), 100, 4, 1499-1504) or a Cr and Au coating by sputtering techniques, and combinations of electroless and electrolytic plating. It should be noted that the inlet channel 13 of the reaction chambers 15 is a continuous compartment providing a fluid communication between sample fluid channels 1 and reaction chambers 15. The lines visible in FIG. 15D illustrate the outlines of the different compartments in the way they are formed from the three substrate layers and do not necessarily represent walls separating them (as e.g. in FIGS. 13 and 14).

FIG. 16 depicts an exemplary use of a device of the present invention in the real-time detection of Dengue viral RNA. The reaction chambers of the device used were preloaded with oligonucleotide primers for the detection of the respective serotypes 1 to 4. The following primers were used:

(a) (all reaction chambers): CAATATGCTGAAACGCGCGAGAAA (SEQ ID NO: 1);
(b) reaction chamber 1 (targeting serotype 1): CGCTCCATACATCTTGAATGAG (SEQ ID NO: 2); reaction chamber 2 (targeting serotype 2): AAGACATTGATGGCTTTTGA (SEQ ID NO: 3); reaction chamber 3 (targeting serotype 3): AAGACGTAAATAGCCCCCGAC (SEQ ID NO: 4); reaction chamber 4, (targeting serotype 4): AGGACTCGCAAAAACGTGATGAAT (SEQ ID NO: 5). RNA was extracted from serum of a subject (140 μl) and suspended in 50 μl water. Using the extracted RNA (1.3 μl), a reaction fluid was prepared (final volume 10 μl), which contained PCR buffer (Invitrogen), DMSO (4%), MgSO4 (4 mM), Sybergreen I dye (2.5 x, Molecular Probes), reverse transcriptase/Taq polymerase (2 μl, Superscript One-Step Sys RT-PCR w/platin, Invitrogen).

After loading and sealing the device as illustrated above the device was placed into a thermal cycling machine (see Example 2) and exposed to the following cycling conditions: 57° C., 30 min (1 cycle); 95° C., 2 min (1 cycle); 40 cycles of 95° C., 10 sec; 57° C., 15 sec; 72° C., 15 sec. FIG. 16 A shows on the right (II) a photo of the respective device taken subsequently. On the left (I) a corresponding device, serving as a negative control, was exposed to the same conditions, wherein the reaction fluid contained sterile water instead of the extracted RNA. The numbering of the reactor modules of the two devices (1 to 4) corresponds to the numbering of the respective reaction chambers used above, and thus to the corresponding serotype. In the embodiment used, each reactor module contained one reaction chamber. The results indicate that the subject whose serum was analysed is infected with Dengue virus subtype 1.

FIG. 16 A depicts the corresponding fluorescent acquisition profiles of the reaction chambers during the course of the reaction. The numbering of the curves (1 to 4) corresponds to the numbering of the respective reaction chambers (see above). The increase of signal intensity in reaction chamber 1 at an earlier time point than the other reaction chambers further indicates the specificity of the binding between the primer used and the extracted RNA.

### Example 1

### Fabrication of a Device for Analysing a Fluid Sample

This example illustrates the fabrication of a device of the invention.

Three pieces of soda lime glass substrate 48 mm×65 mm×0.17 mm were obtained from Erie Scientific (USA). The glass substrates were cleaned with piranha solution (H₂O₂:H₂SO₄, 1:2 ratio) according to published procedures. The glass substrates were dehydrated at 100° C. before they were coated with 20 nm of Cr and 80 nm of Au inside a high vacuum electron beam machine. The same Cr and Au coating process can be achieved using alternative standard processes such as sputtering techniques, and combinations of electroless and electrolytic plating.

The metal-coated glass pieces were coated with a photoresist on both sides. The desired micro-fluidics patterns were then formed using standard photolithographic techniques. The exposed pattern of Cr and Au layers were removed using commercially available chrome etching solution and gold etching solution to form the sacrificial patterns prior to glass etching. The photoresist was then stripped using acetone.

The glass substrates with patterned Cr and Au layers were then subjected to Hydrofluoric Acid solution to etch the glass to form the micro-fluidics channels. It should be noted that the depth of the channels and loading ports depends on the required functions and applications of the chips. In the present example the channels and loading ports were etched up to 100 μm. The sacrificial layers of Cr and Au were then removed using the same chemicals as above.

An illustrative top view of the microfluidic structures for each of the three substrate layers is shown in FIG. 15.

The first layer, second layer and the third layer substrate (cf. FIG. 15A to 15C) were brought together visually and aligned and bonded to form the device (cf. FIG. 15D).

### Example 2

### PCR Assay with Reaction Chambers Coated with Sybergreen I

This example illustrates the use of the device of the invention for PCR Assay with Sybrgreen in reaction chambers of the dimensions 2.1×1.4×0.2 mm. The embodiment of the device used was a micro chip with the dimensions 48×65 mm (width×length), wherein each reactor module contained one reaction chamber. The embodiment of the device resembled the one depicted in FIGS. 12 and 15, containing four reactor modules.

Total RNA was extracted from 140 μl serum of a subject using the Qiamp Viral RNA mini kit (Qiagen). The RNA was eluted in a volume of 50 μl sterile water.

During fabrication, the device was preloaded with oligonucleotide primers for the detection of the respective serotypes 1 to 4. Primers were deposited by discrete spotting of aliquots of onto the surface of each reaction chamber. The forward primer used in all reaction chambers was 5′-CAATATGCTGAAACGCGCGAGAAA-3′ (SEQ ID No: 1). The reverse primers used were:

| | | |
|---|---|---|
| Reactor | | |
| module | Target | |
| Number | Sero-type | Primer 2 |

| | | | |
|---|---|---|---|
| 1 | 1 | 5′-CGCTCCATACATCTTGAATGAG-3′ | (SEQ ID NO: 2) |
| 2 | 2 | 5′-AAGACATTGATGGCTTTTGA-3′ | (SEQ ID NO: 3) |
| 3 | 3 | 5′-AAGACGTAAATAGCCCCCGAC-3′ | (SEQ ID NO: 4) |
| 4 | 4 | 5′-AGGACTCGCAAAAACGTGATGAAT-3′ | (SEQ ID NO: 5) |

For primers 2 and 3 the final concentration in the reaction chamber when resuspended was 0.2 μM. For primers 3 and 4 it was 0.15 μM. After drying at 45 deg C. for 30 minutes, the devices were bonded prior to use.

For each device sample 10 μl fluid sample were prepared, which contained RT-PCR buffer (1×, Invitrogen), MgSO₄ (4 mM), DMSO (4% v/v), BSA (0.5 mg/ml), Sybergreen I dye (2.5×, Molecular Probes), reverse transcriptase/Taq polymerase (2 μl, Superscript One-Step Sys RT-PCR w/platin, Invitrogen), RNA (1.3 μl). In a control device, the above fluid sample had the same composition, with the exception that sterile water (1.3 μl) was used to replace the RNA.

8.5 μl of the above fluid sample were introduced into loading port 5 of the micro chip (cf. FIG. 15D or 10A) and allowed to diffuse into the reaction chambers. Subsequently 15 μl of silicone RTV were inserted as a sealing material into loading ports 5 and 7 to seal the sample transmission channel 1 and the multi-functional channel 3.

Afterwards, the device was placed into a compatible real-time thermal cycling machine (Attocycler, Attogenix Biosystems Pte Ltd) and subjected to the following PCR conditions: 57° C., 30 min (1 cycle); 95° C., 2 min (1 cycle); followed by 40 cycles of 95° C., 10 sec; 57° C., 15 sec; 72° C., 15 sec. The results, which are shown in FIGS. 16A and 16B, show positive detection of dengue virus subtype 1 present in the RNA extracted from the subject (reaction chamber 1). This serotype correlated with the serology tests carried out on the same subject. Furthermore, the fluorescent acquisition profiles of the reaction chambers during the course of the reaction, depicted in FIG. 16B, indicated that the binding between the primer used and the extracted RNA was specific.

### Example 3

### Antibody-Antigen Fluorescence Quenching

This example illustrates the use of the device of the invention for antibody-antigen fluorescence quenching assay in reaction chambers of the dimensions 2.1×1.4×0.2 mm. An antibody was labelled with OG-514 (Oregon green 514 carboxylic acid, succinimidyl esters) and an antigen (peptide, polypeptide, protein, whole cells, carbohydrate, aptamers, etc.) was labelled with QSY-7 (QSY-7 carboxylic acid, succinimidyl esters). Fluorescence quenching prevented or suppressed the detection of OG-514 fluorescence. The labelled antibody-antigen complex was disposed in the reaction chamber(s) in lyophilized form. The introduction of fluid sample rehydrated and dissolved the complex in the respective PBS or TBS buffer with or without detergent (e.g. Tw-20 or Triton-X 100) of various concentration (e.g. 0.05% Tw-20 and 1% Triton-X-100). Upon re-hydration, the antigen in the labelled antibody-antigen complex competed with introduced unlabeled antigen, contained in the fluid sample. Competition with unlabeled antigen released the OG-514 labelled antibody the fluorescence of which was detected at about 528-530 nm n.

1-69. (canceled)

70. A microfluidic device for analysing a fluid sample, comprising:
at least one sample transmission channel;
at least one multi-functional channel; and
at least one reactor module fluidly connecting the sample transmission channel to the multi-functional channel, said at least one reactor module comprising
at least one reaction chamber having at least one inlet in fluid communication with the at least one sample transmission channel, and
at least one fluid isolation chamber, the fluid isolation chamber being in fluid communication with at least one outlet of the at least one reaction chamber; wherein
the fluid isolation chamber isolates the fluid sample from the at least one multi-functional channel.

71. The microfluidic device according to claim 70, wherein the fluid isolation chamber is arranged to provide physical separation between the fluid sample and a sealing material introduced into the multi-functional channel, the fluid isolation chamber being in fluid communication with the multi-functional channel.

72. The microfluidic device according to claim 71, wherein the fluid isolation chamber is connected to the multi-functional channel via an outlet, the outlet of the reaction chamber being in fluid communication with an inlet of the fluid isolation chamber, which is located on a wall opposing the outlet of the fluid isolation chamber.

73. The microfluidic device according to claim 72, wherein the outlet of the reaction chamber is connected to the inlet of the fluid isolation chamber via an inclined port.

74. The microfluidic device to claim 73, wherein the angle formed between a base of the fluid isolation chamber and a lateral wall of the port is within a range from 0° to 180°.

75. The microfluidic device according to claim 74, wherein the angle formed between the base of the fluid isolation chamber and the lateral wall of the port is within range from 45° to 135°.

76. The microfluidic device according to claim 75, wherein the lateral wall of the port is perpendicular to the base of the fluid isolation chamber.

77. The microfluidic device according to claim 70, wherein the at least one outlet of the reaction chamber comprises at least one microcapillary channel, an opening of the least one micro-capillary channel providing fluid communication with the fluid isolation chamber, wherein the diameter of the opening is smaller than the diameter of the microcapillary channel.

78. The microfluidic device according to claim 77, wherein the diameter of the opening is about 1.5-fold to about 20-fold smaller than the diameter of the microcapillary channel.

79. The microfluidic device to claim 78, wherein the diameter of the opening is about 2-fold to about 10-fold smaller than the diameter of the microcapillary channel.

80. The microfluidic device according to claim 79, wherein the diameter of the opening is about 3-fold to about 6-fold smaller than the diameter of the microcapillary channel.

81. The microfluidic device according to claim 80, wherein the at least one outlet of the reaction chamber comprises two microcapillary channels located at a distal portion of the reaction chamber with respect to the inlet of the reaction chamber.

82. The microfluidic device according to claim 77, wherein the at least one micro capillary channel is situated at a position intermediate the reaction chamber and the fluid isolation chamber.

83. The microfluidic device according to claim 70, wherein the reaction chamber comprises a convex shape reaction chamber wall adjacent the at the least one outlet of the reaction chamber.

84. The microfluidic device according to claim 83, wherein the convex shape is selected from the group consisting of: hemispherical, semi-elliptical, polygonal protrusion, and at least one irregularly shaped protrusion.

85. The microfluidic device according to claim 70, wherein the reactor module has a shape selected from the group consisting of: a rectangle, square, ovoid and bottle-shape.

86. The microfluidic device according to claim 70, wherein at least one of: surface characteristics and geometrical characteristics, are used for regulating the conduction of the fluid sample.

87. The microfluidic device to claim 86, wherein the surface characteristics are provided for by a coating.

88. The microfluidic device according to claim 88, wherein the coating comprises a compound selected from the group consisting of hexamethyldisilazane, trimethylchlorosilane, dimethyldichlorosilane, propyltrichlorosilane, tetraethoxysilane, glycidoxypropyltrimethoxysilane, 3-aminopropyltriethoxysilane, 2-(3,4-epoxy cyclohexyl)ethyltrimethoxysilane, 3-(2-3-epoxy propoxy)propyltrimethoxysilane, poly-dimethysiloxane (PDMS), γ-(3,4-epoxycyclohexyl)ethyltrimethoxysilane, poly(methyl methacrylate), urethane, polyurethane, fluoropolyacrylate, poly(methoxy polyethylene glycol methacrylate), poly(dimethyl acrylamide), poly[N-(2-hydroxypropyl)methacrylamide] (PHPMA), α-phosphorylcholine-o-(N,N-diethyl-dithiocarbamyl)undecyl oligo DMAAm-oligo-STblock co-oligomer, 3,4-epoxy-cyclohexylmethylmethacrylate, 2,2-bis[4-(2,3-epoxypropoxy)phenyl]propane, 3,4-epoxy-cyclohexylmethylacrylate, (3′,4′-epoxycyclohexymethyl)-3,4-epoxy-cyclohexyl carboxylate, di-(3,4-epoxycyclohexylmethyl)adipate, bisphenol A (2,2-bis-(p-(2,3-epoxy propoxy)phenyl)propane) and 2,3-epoxy-1-propanol.

89. The microfluidic device to claim 86, wherein the walls of the transmission channel have a lower affinity for the fluid sample than the walls of the reaction chamber, and the walls of the multi-functional channel have a lower affinity for the fluid sample than the walls of the fluid isolation chamber.

90. The microfluidic device according to claim 70, wherein at least one wall of the reaction chamber is coated with a compound for carrying out an assay reaction to analyse a property of the fluid sample.

91. The microfluidic device according to claim 70, wherein the reaction chamber is sealed against at least one of said transmission channel or multi-functional channel by a sealing material.

92. The microfluidic device according to claim 91, wherein the sealing material is a solid that is activated by one of: mechanically, electrically, magnetically and hermetically.

93. The microfluidic device according to claim 92, wherein the sealing material comprises at least one selected from the group consisting of: a polymer in a gel state, a polymer in a liquid state, a polymer derived from a photo-sensitive polymer pre-cursor, and a polymer derived from a heat-sensitive polymer pre-cursor.

94. The microfluidic device according to claim 92, wherein the sealing material comprises a visually-active pigment selected from the group consisting of: carbon pigments, organic dyes and fluorescent dyes.

95. The microfluidic device according to claim 70, wherein the reactor module is etched onto a substrate comprising a material selected from the group consisting of: silicon, quartz, glass, plastic, elastomer, metal and composites thereof.

96. The microfluidic device according to claim 70, further comprising a covering layer.

97. The microfluidic device according to claim 96, wherein at least a part of the covering layer comprises a self-sealing material.

98. The microfluidic device according to claim 70, further comprising a plurality of reactor modules, each reactor module fluidly connecting the sample transmission channel to the multi-functional channel.

99. A method of detecting an analyte in a fluid sample, comprising:
(a) providing a microfluidic device for detecting an analyte in a fluid sample, comprising:
at least one sample transmission channel;
at least one multi-functional channel; and
at least one reactor module fluidly connecting the sample transmission channel to the multi-functional channel, said at least one reactor module comprising:
at least one reaction chamber having at least one inlet in fluid communication with the at least one sample transmission channel, and
at least one fluid isolation chamber, the fluid isolation chamber being in fluid communication with at least one outlet of the at least one reaction chamber, wherein the fluid isolation chamber isolates the fluid sample from the multi-functional channel;
(b) loading the fluid sample into said microfluidic device,
(c) sealing the at least one sample transmission channel and the at least one multi-functional channel with a sealing material, and
(d) carrying out at least one analyte detection reaction, said reaction, providing at least one qualitative or quantitative datum relating to the analyte.

100. A method according to claim 99, wherein the loading is by introducing the fluid sample into the sample transmission channel, the volume of fluid sample introduced into the sample transmission channel being selected to be substantially equal to or less than the combined volume of said at least one reaction chamber.

101. The method according to claim 100, wherein there is a plurality of reactor modules.

102. The method according to claim 101, wherein the plurality of reactor modules are filled with the fluid sample in a manner selected from the group consisting of: simultaneously, and sequentially.

103. The method according to claim 102, wherein the plurality of reactor modules are partially filled with the fluid sample.

104. The method according to claim 99, wherein the at least one outlet of the reaction chamber comprises at least one microcapillary channel, the fluid sample is not being distributed into the least one microcapillary channel.

105. The method according to claim 99, wherein said sealing comprises introducing the seal material into at least one of: the sample transmission channel, and the multi-functional channel.

106. The method according to claim 105, wherein the sealing material displaces a portion of the fluid sample into the at least one microcapillary channel.

107. The method according to claim 99, wherein the sealing material comprises a polymer precursor.

108. The method according to claim 107, further comprising polymerizing the polymer precursor, thereby forming a polymer.

109. The method according to claim 99, wherein said at least one qualitative or quantitative datum provides at least one result selected from the group consisting of: colorimetric, fluorometric and luminescent results.

110. The method according to claim 109, wherein said fluorometric result is derived from fluorescence provided by at least one of: binding of a fluorophore and hybridization of a probe containing a fluorophore; and said at least one qualitative or quantitative data is obtained via a probe labeled with at least one of a fluorophore, an enzyme, or component of a binding complex.

111. The method according to claim 99, wherein the fluid sample comprises biological material comprising at least one analyte selected from the group consisting of: metabolites, nucleotides, polynucleotides, nucleic acids, amino acids, peptides, polypeptides, proteins, biochemical compositions, lipids, carbohydrates, cells, and microorganisms.

112. The method according to claim 99, wherein the fluid sample comprises non-biological material comprising at least one analyte selected from the group consisting of: ions, synthetic compounds, organic chemical compositions, inorganic chemical compositions, combinatory chemistry products, drug candidate molecules, drug molecules, drug metabolites, and any combination thereof.

113. The method according to claim 99, wherein said at least one analyte detection reaction comprises at least one selected from the group consisting of: a nucleic acid amplification, an immunodetection reaction, and an Enzyme-Linked Immunosorbent Assay.

114. The method according to claims 99, wherein said method is carried out to determine a property of the fluid sample, said property being selected from the group consisting of analyte quantities, reaction kinetic constants, affinity constants, analyte purity, and analyte heterogeneity.
